SEQUENCE LISTING (10 sequences)

SEQ ID NO: 1 - DNA, 17 bp, Artificial Sequence
  Description of Artificial Sequence Synthetic oligonucleotide
aagttggttggtttgtt

SEQ ID NO: 2 - DNA, 16 bp, Artificial Sequence
  Description of Artificial Sequence Synthetic oligonucleotide
gttggtttgttacctg

SEQ ID NO: 3 - DNA, 16 bp, Artificial Sequence
  Description of Artificial Sequence Synthetic oligonucleotide
accaaccaactttcga

SEQ ID NO: 4 - DNA, 14 bp, Artificial Sequence
  Description of Artificial Sequence Synthetic oligonucleotide
ttggttggtttgtt

SEQ ID NO: 5 - DNA, 17 bp, Artificial Sequence
  Description of Artificial Sequence Synthetic oligonucleotide
ccaactttcgatctctt

SEQ ID NO: 6 - DNA, 17 bp, Artificial Sequence
  Description of Artificial Sequence Synthetic oligonucleotide
aacaaaccaaccaactt

SEQ ID NO: 7 - DNA, 16 bp, Artificial Sequence
  Description of Artificial Sequence Synthetic oligonucleotide
caggtaacaaaccaac

SEQ ID NO: 8 - DNA, 16 bp, Artificial Sequence
  Description of Artificial Sequence Synthetic oligonucleotide
tcgaaagttggttggt

SEQ ID NO: 9 - DNA, 14 bp, Artificial Sequence
  Description of Artificial Sequence Synthetic oligonucleotide
aacaaaccaaccaa

SEQ ID NO: 10 - DNA, 17 bp, Artificial Sequence
  Description of Artificial Sequence Synthetic oligonucleotide
aagagatcgaaagttgg

(19) United States
(12) Patent Application Publication
(10) Pub. No.: US 2023/0242588 A1
(43) Pub. Date: Aug. 3, 2023

(54) METHODS AND COMPOSITIONS FOR TREATING SARS-COV-2 INFECTION USING PEPTIDE NUCLEIC ACID-BASED AGENTS

(71) Applicant: Oncogenuity, Inc., New York, NY (US)

(72) Inventors: Arthur J. Ross, New York, NY (US); Lindsay Rosenwald, New York, NY (US)

(21) Appl. No.: 17/926,095

(22) PCT Filed: May 17, 2021
(86) PCT No.: PCT/US2021/032790

§ 371 Date: Nov. 17, 2022

Related U.S. Application Data
(60) Provisional application No. 63/026,601, filed on May 18, 2020.

(51) Int. Cl. C07K 14/00; A61P 31/14

(52) U.S. Cl. CPC C07K 14/003; A61P 31/14

(57) ABSTRACT

The invention relates generally to viral infections and more specifically to compositions and methods for treating infections by SARS-CoV-2 (2019-nCoV). In particular, the invention provides a PNA agent which includes a PNA moiety with a sequence that targets a SARS-CoV-2 gene; a first cationic and hydrophobic peptide at the N-terminus of the PNA moiety, wherein the first peptide comprises lysine residues, and at least one of the lysine residues comprises a palmitoyl side chain moiety; and a second cationic and hydrophobic peptide at the C-terminus of the PNA moiety, wherein the second peptide comprises lysine residues, and at least one of the lysine residues comprises a palmitoyl side chain moiety.

### CROSS-REFERENCE TO RELATED APPLICATIONS

This application claims the benefit of priority under 35 U.S.C. § 119(e) of U.S. Provisional Application No. 63/026,601, filed May 18, 2020. The contents of the prior application is considered part of and is incorporated by reference in the disclosure of this application.

### INCORPORATION OF SEQUENCE LISTING

The material in the accompanying sequence listing is hereby incorporated by reference into this application. The accompanying sequence listing text file, named OG1100_1WO_SL.txt was created on May 17, 2021, and is 11 kb. The file can be assessed using Microsoft Word on a computer that uses Windows OS.

### BACKGROUND OF THE INVENTION

### Field of the Invention

The invention relates generally to viral infections and more specifically to compositions and methods for treating infections by SARS-CoV-2.

### Background Information

Coronaviruses (CoVs) are a large group of viruses that commonly originate in many different species of animals. Some coronaviruses can make a jump from animals to human, and cause the respiratory infections including mild and cold-like symptoms. A couple of coronaviruses including Middle East respiratory syndrome (MERS) and severe acute respiratory syndrome (SARS), are much more severe and have killed thousands of people.

2019-nCoV, officially named as SARS-CoV-2, is another contagious and novel coronavirus that can infect people and cause outbreak of respiratory illness (COVID-19). It was first detected in Wuhan, China in December 2019, and is being reported in a growing number of international locations. The rapid person-to-person spread of SARS-CoV-2 presents an imminent threat to the global public health. It was declared as pandemic by WHO on Mar. 11, 2020. As of Apr. 30, 2021, over 150,751,023 COVID-19 cases caused by SARS-CoV-2 have been recorded worldwide, and more than 3,170,271 people have been reported dead.

Chinese health authorities were the first to isolate the SARS-CoV-2, the Wuhan seafood market pneumonia virus isolate Wuhan-Hu-1, and published the full genome sequence of the SARS-CoV-2 (2019-nCoV) which is ˜90% of nucleotide similarity to a group of SARS-like coronaviruses. Protein sequence analysis revealed that 2019-nCoV shares ˜80% sequence identity to SARS-CoV, and 96% identity to a bat coronavirus at the whole-genome level. US CDC has also posted the full genome of the SARS-CoV-2 viruses detected in U.S. patients, and found the sequences from US patients similar to the one that China initially posted.

Like MERS and SARs, SARS-CoV-2 are also large, enveloped, positive-sense, single-stranded RNA viruses. The genome of coronavirus encodes four major structural proteins including Spike (S) protein, Nucleocapsid (N) protein, Envelope (E) protein, and Membrane (M) protein, as well as a number of accessory open reading frame (ORF) proteins.

The coronavirus Nucleocapsid (N) is a structural protein of multifunction. The N protein of CoVs forms the helical ribonucleocapsid complexes with positive strand viral genomic RNA, and interacts with viral membrane protein during virion assembly, and plays an important role in enhancing the efficiency of virus replication, transcription, and assembly.

The coronavirus Spike protein (S) is a large oligomeric transmembrane protein that mediates coronavirus entry into host cells. It contains S1 and S2 two subunits. Spike S1 mainly contains a receptor binding domain (RBD) that recognizes a variety of host cell surface receptors. S2 contains basic elements responsible for the membrane fusion. The coronavirus first binds to a receptor on the host cell surface through Spike S1 subunit, and then fuses viral and host membranes through Spike S2 subunit.

Structural modelling studies of Spike proteins from SARS-CoV-2 and SARS-CoVs suggests that the SARS-CoV-2 S protein retains sufficient affinity to the cellular Angiotensin converting enzyme 2 (ACE2) protein, and likely uses ACE2 protein as a receptor for cellular entry. Most recent studies show that the 2019-nCoV S's affinity to bind to ACE2 is 10-20-fold higher than that of SARS-CoV S.

A SARS-CoV-2 virion is approximately 50-200 nanometers in diameter. Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the complete viral envelope. The spike protein, S, which has been imaged at the atomic level using cryogenic electron microscopy, is the protein responsible for allowing the virus to attach to and fuse with the membrane of a host cell. As used herein, the phrase SARS-CoV-2 structural “protein S, N, M, and/or E” refers to the spike (S), nucleocapsid (N), membrane (M), and/or envelope (E) proteins, respectively. The nucleic acid sequence can include a codon-optimized oligonucleotide sequence encoding each protein individually, or any combination of 2 or 3 proteins, or a combination of all 4 proteins. When two or more nucleic acid sequences are included in a single vector or construct, they are in operable linkage such that the each of the 2, 3, or 4 SARS-CoV-2 structural proteins are properly encoded and expressed. (Zhou, P. et al. A pneumonia outbreak associated with a new coronavirus of probable bat origin. Nature doi.org/10.1038/s41586-020-2012-7 (2020); Wu, F. et al. A new coronavirus associated with human respiratory disease in China. Nature doi.org/10.1038/s41586-020-2008-3 (2020); Letko, Michael; Munster, Vincent (22 Jan. 2020). “Functional assessment of cell entry and receptor usage for lineage B β-coronaviruses, including 2019-nCoV”. BiorXiv: 2020.01.22.915660. doi:10.1101/2020.01.22.915660; Wrapp, D.; Wang, N. et al. (19 Feb. 2020). “Cryo-EM structure of the 2019-nCoV spike in the prefusion conformation”. Science science.sciencemag.org/content/early/2020/02/19/science.abb2507 (02/19/2020)).

Coronavirus RNA-dependent RNA synthesis includes two differentiated processes: genome replication, yielding multiple copies of genomic RNA (gRNA), and transcription of a collection of sgmRNAs that encode the viral structural and accessory proteins (Enjuanes L, Almazan F, Sola I, Zuñiga S. Biochemical aspects of coronavirus replication and virus-host interaction. Annu Rev Microbiol. 2006; 60:211-30; Lai MMC, Cavanagh D. The molecular biology of coronaviruses. Adv Virus Res. 1997; 48:1-100).

Like that of other positive-strand RNA viruses, coronavirus genome replication is a process of continuous synthesis that utilizes a full-length complementary negative-strand RNA as the template for the production of progeny virus genomes. The initiation of negative-strand synthesis involves access of the RNA-dependent RNA polymerase (RdRp) to the 3′ terminus of the genome, promoted by 3′-end RNA sequences and structures. There is evidence that both 5′- and 3′-end RNA elements are required for the production of progeny positive-strand RNA from the intermediate negative-strand RNA, suggesting that interactions between the 5′ and 3′ ends of the genome contribute to replication (Sola I, Mateos-Gomez P A, Almazan F, Zuñiga S, Enjuanes L. RNA-RNA and RNA-protein interactions in coronavirus replication and transcription. RNA Biol. 2011; 8:237-48).

While there are many antiviral drugs and vaccines in clinical trials, to date, there have been no effective therapies for infection by SARS-CoV-2 beside vaccination.

### SUMMARY OF THE INVENTION

The present invention relates to compositions for treating infection with coronavirus, and in particular SARS-CoV-2, which is responsible for the respiratory illness, COVID-19. Among other things, the present invention recognizes the source of a problem with conventional antiviral treatments and provides the insight that compounds which target particular viral gene expression, as described herein, are particularly useful in various contexts for treating SARS-CoV-2 infection.

Among other things, the present disclosure demonstrates that peptide nucleic acid (PNA) agents that can specifically target genes, can enable improved treatments for SARS-CoV-2 infection. In some embodiments, use of peptide nucleic acid agents will enable improved methods for suppressing and treating SARS-CoV-2 infection within a clinical setting. Furthermore, peptide nucleic acids with terminal cationic and/or hydrophobic moieties that improve solubility within cell membranes can be more effective at crossing cellular membranes than previous PNA-type agents and offer stabilization toward the anionic chromosomal target. U.S. Pat. No. 10,113,169 provides the background for PNA molecules useful for the present invention and is herein incorporated by reference in its entirety.

PNA agents are promising tools in the research and development of new drugs to treat diseases such as SARS-CoV-2 infection. The present disclosure provides improved PNA agents, as well as technologies for designing, identifying, characterizing and/or using them, and compositions that include them.

Among other things, the present invention encompasses the recognition that one unmet need in use of available PNA-based drugs is the successful delivery of agents across cellular membranes to the target gene. For example, the ability to cross cell membranes is mediated by cationic/hydrophobic delivery peptides. Among other things, the present invention discloses PNA agents whose physio-chemical properties contribute to improved drug delivery across cell membranes (e.g., relative to available PNA agents).

For example, the present disclosure demonstrates that cationically charged termini on PNA agents improve the ability to target non-promoter regions of genes, which are less open and exposed compared to promoter regions. Without wishing to be bound by any particular theory, the present disclosure proposes that stabilizing the cationically charged lysine-derivatized PNA termini against the anionic DNA improves a PNA's binding kinetics to targets. These terminal modifications allow stabilization of the conjugate termini towards the chromosomal anionic phosphate esters due to cationic-anionic interaction. This aids a PNA's strand-invading properties and allows it to displace the complementary strand of its SARS-CoV-2 gene target.

PNA agents of the present disclosure are modified relative to traditional PNA agents through use of cationic/hydrophobic peptides; in some embodiments, such modified PNA agents show improved delivery across cell membranes relative to that observed with otherwise comparable PNA agents that do not include such cationic/hydrophobic peptides. Again, without wishing to be bound by any particular theory, the present disclosure proposes that hydrophobic and cationic terminal peptides together facilitate passive transport of inventive PNA agents across membranes. For example, in certain particular embodiments, hydrophobic e-palmitoyl lysine termini are driven together by solvent exclusion, and the PNA-peptide conjugate is intramolecularly further stabilized by pi-interacting nucleoside bases. In some embodiments, a PNA-peptide chain is compacted (i.e., displays a decreased radius of gyration) through such interactions, allowing it to more easily permeate a membrane (e.g., a lipid bilayer). Furthermore, it is hypothesized that cationic-anionic interactions between PNA agents of the present invention and phospholipids cell membrane also facilitate the PNA agent's insertion into the membrane.

With respect to PNA agents, there is a thermodynamic equilibrium between stable binding within a cell membrane and dissociating from the membrane. Without wishing to be bound by any particular theory, it is envisioned that the modifications of PNA agents of the present invention ease the kinetic barrier for membrane insertion of PNA agents so that the aforesaid equilibrium is more quickly achieved. This, in turn, accelerates transmembrane transport.

Without wishing to be bound by any particular theory, it is contemplated that a PNA-agent of the present invention exists in an equilibrium between folded and open states. The folded state lends itself well for cell membrane insertion, and the open-coil state lends itself well for helical association with chromosomal targets for a better facilitated helix-coil transition.

The art has developed a variety of strategies for transporting oligonucleotides across cell membranes. The present invention provides improved systems, permitting enhanced transport of provided PNA derivatives across cell membranes and intracellular delivery, and furthermore facilitating binding of PNA agents to and targeting of less exposed regions of DNA.

Embodiments of the present invention encompass the surprising discovery that peptide nucleic acid agents with modified termini as described herein can better cross cellular membranes and bind to target genes. According to some embodiments of the present invention, modified peptide nucleic acid agents specific for regions RNA encoding SARS-CoV-2 structural proteins, such as S, N, M, or E, can suppress transcription and ultimately translation of these viral proteins, as well reduce viability of cells expressing the proteins, e.g., virus-infected cells.

Embodiments of the present invention encompass the surprising discovery that peptide nucleic acid agents can bind to genes associated with a viral infection.

In one embodiment, the invention provides a nucleic acid sequence selected from SEQ ID NO:1-10. In one aspect, the sequence is a targeting sequence selected from SEQ ID NO:1-5. Such nucleic acid sequences can be incorporated into a vector for delivery, including plasmid or viral vectors, or the PNAs of the invention.

In one embodiment, the invention provides a PNA agent including a PNA moiety including a nucleic acid sequence that targets a SARS-CoV-2 nucleic acid sequence; a first cationic and hydrophobic peptide at the N-terminus of the PNA moiety, wherein the first peptide includes lysine residues; and a second cationic and hydrophobic peptide at the C-terminus of the PNA moiety, wherein the second peptide includes lysine residues.

In one aspect, at least one of the lysine residues include a palmitoyl side chain moiety. In another aspect, the PNA agent includes a sequence that has minimal propensity to form hairpin loops. In some aspects, the PNA agent includes a sequence that contains less than 60% purines. In other aspects, the PNA moiety includes a sequence that targets an RNA sequence of SARS-CoV-2 virus. In one aspect, the RNA sequence is a positive or a negative RNA strand of the SARS-CoV-2 virus. In some aspects, the PNA moiety includes a sequence selected from SEQ ID NO:1, SEQ ID NO:2, SEQ ID NO:3, SEQ ID NO:4 and SEQ ID NO:5. In other aspects, the PNA moiety includes a nucleic acid sequence that targets an RNA sequence selected from the group consisting of SEQ ID NO:6, SEQ ID NO:7, SEQ ID NO:8, SEQ ID NO:9 and SEQ ID NO:10. In one aspect, the PNA moiety includes a sequence that targets a 13-20 nucleotide sequence with about 75% or greater complementarity to the targeting moiety. In another aspect, the PNA moiety includes a sequence that targets a 13-20 nucleotide sequence with complete complementarity. In one aspect, the nucleic acid sequence is a SARS-CoV-2 nucleic acid sequence encoding a structural protein or an open reading frame (orf) protein. In another aspect, the SARS-CoV-2 nucleic acid sequence encodes a structural protein selected from a Spike (S) protein, Nucleocapsid (N) protein, Envelope (E) protein, or Membrane (M) protein. In some aspects, the PNA agent targets a nucleic acid sequence encoding the S protein.

In another embodiment, the invention provides a pharmaceutical composition including any one of the PNA agents described herein and pharmaceutically acceptable carrier.

In an additional embodiment, the invention provides a method for treating or reducing the risk of a SARS-CoV-2 infection including administering to a subject susceptible to or having a SARS-CoV-2 infection any one of the PNA agents described herein or any one of the pharmaceutical compositions described herein.

In one aspect, the subject has COVID-19. In another aspect, the PNA agent includes a PNA moiety including a sequence selected from SEQ ID NO:1, SEQ ID NO:2, SEQ ID NO:3, SEQ ID NO:4 and SEQ ID NO:5. In some aspects, the PNA agent includes a moiety including a nucleic acid sequence that targets an RNA sequence selected from the group consisting of SEQ ID NO:6, SEQ ID NO:7, SEQ ID NO:8, SEQ ID NO:9 and SEQ ID NO:10. In other aspects, the PNA agent increases the viability of a cell infected by SARS-CoV-2 in the subject. In some aspects, the PNA agent reduces SARS-CoV-2-associated cytopathic effects in a cell infected by SARS-CoV-2 in the subject. In other aspects, the PNA agent inhibits SARS-CoV-2-associated cytopathic effects with a IC50 that is less than about 5 μM. In one aspect, the PNA agent inhibits SARS-CoV-2-associated cytopathic effects by at least 50%. In other aspects, the method further includes administering an anti-viral agent. In some aspects, the anti-viral agent is Remdesivir.

In one embodiment, the invention provides a method of reducing expression of a SARS-CoV-2 target nucleic acid sequence in a cell including contacting a cell in which the target is expressed with at least one of the PNA agents described herein, determining a level or activity of the target in the cell when the PNA agent is present as compared with a target level or activity observed under otherwise comparable conditions when the PNA agent is absent; and classifying the PNA agent as a target inhibitor if the level or activity of the target is reduced when the PNA agent is present as compared with the target level or activity in the absence of the PNA agent, thereby reducing expression of the SARS-CoV-2 target nucleic acid sequence.

In one aspect, the method further includes detecting a viral load of SARS-CoV-2.

In another embodiment, the invention provides a method for identifying and/or characterizing a PNA agent as an inhibitor of a target nucleic acid sequence including contacting a SARS-CoV-2 target nucleic acid sequence with at least one PNA agent; determining a level or activity of the target sequence in a system when the PNA agent is present as compared with a target reference level or activity under otherwise comparable conditions when is the PNA agent is absent; and classifying the PNA agent as a target inhibitor if the level or activity of the target is reduced when the PNA agent is present as compared with the target level or activity when the PNA agent is absent, thereby identifying and/or characterizing the PNA agent as an inhibitor or a target nucleic acid sequence.

In one aspect, determining the level or activity of the target includes determining a target RNA level of expression. In another aspect, determining the level or activity of the target includes determining a target protein level. In one aspect, the system includes an in vitro system. In another aspect, the system includes an in vivo system. In some aspects, the system includes cells. In one aspect, the level or activity of the target corresponds to cell viability. In some aspects, a reduction in the level or activity of the target corresponds to a greater than about 90% increase in cell viability. In various aspects, the cells include SARS-CoV-2 virus. In other aspects, the system includes cells in cell culture. In some aspects, the system includes a tissue. In other aspects, the system includes an organism. In another aspect, the level or activity of the target corresponds to survival of the organism. In some aspects, a reduction in the level or activity of the target includes a greater than 50% increase in survival of the cell or organism. In one aspect, the organism includes a non-human mammal or a human. In another aspect, a reduction in the level or activity of the target includes a greater than 50-100% reduction of target activity. In various aspects, a reduction in the level or activity of the target includes a greater than 30% reduction of target levels.

In some embodiments, the invention provides PNA agents including a PNA moiety; a first cationic or hydrophobic moiety at a first end of the PNA moiety; and a second cationic or hydrophobic moiety at a second end of the PNA moiety. In some embodiments, the first cationic moiety is or includes a peptide. In some embodiments, the first cationic peptide includes or consists of lysine residues. In some embodiments, at least one lysine residue includes a palmitoyl side chain moiety. In some embodiments, the first cationic peptide includes amines.

In some embodiments, the invention provides PNA agents including a PNA moiety; a first cationic moiety and a first hydrophobic moiety at a first end of the PNA moiety; and a second cationic moiety and a second hydrophobic moiety at a second end of the PNA moiety. In some embodiments, the first cationic and/or hydrophobic moiety is or includes a peptide. In some embodiments, the second cationic and/or hydrophobic moiety is or includes a peptide. In some embodiments, the first and/or second cationic peptide includes one or more lysine residues. In some embodiments, the first and/or second hydrophobic peptide includes one or more lysine residues. In some embodiments, at least one lysine residue includes a palmitoyl side chain moiety. In some embodiments, at least one lysine residue at either end of the PNA moiety includes a palmitoyl side chain moiety. In some embodiments, the first and/or second cationic and/or hydrophobic peptide includes amines.

In some embodiments, the palmitoyl lysine is not attached to the PNA moiety directly, but via one or more additional amino acids.

In some embodiments, the PNA agent has a sequence that does not form hairpin loops. In some embodiments, the PNA agent has a sequence that has a tendency to not form hairpin loops. In some embodiments, the PNA agent has a sequence that contains less than 60% purines. In some embodiments, the first and/or second cationic and/or hydrophobic moiety is a targeting moiety in that the terminal cationic moieties more effectively align themselves with the DNA anionic phosphoribose. This allows them a greater statistical likelihood of finding the nucleic acid with the sequence to which they are targeted. The terminal hydrophobic/cationic residues more effectively ease the PNA derivative through cell membranes. The termini likely associate intramolecularly by ‘hydrophobic solvent exclusion,’ thus increasing the statistically high likelihood of the termini being within proximity of each other. In some embodiments, the second cationic or hydrophobic moiety is or includes a cationic and/or hydrophobic peptide.

In some embodiments, the targeting moiety is at the PNA agent's N-terminus. In some embodiments, the cationic peptide is at the PNA agent's C-terminus. In some embodiments, the first cationic or hydrophobic moiety is a targeting moiety in that the terminal cationic moieties more effectively align themselves with the DNA anionic phosphoribose to allow a greater statistical likelihood of finding the nucleic acid with the sequence to which they are targeted and is attached at the PNA agent's N-terminus; and the second cationic or hydrophobic moiety is or includes a cationic peptide that is attached at the PNA agent's C-terminus.

In some embodiments, PNA agents include a sequence that targets a nucleic acid sequence. In some embodiments, PNA agents include a sequence that targets a 13-20 nucleotide sequence of a nucleic acid sequence, e.g., SARS-CoV-2 nucleic acid sequence, with 75% or greater complementarity. In some embodiments, PNA agents include a nucleic acid whose length is at least 14, 15, 16, 17, or 18 nucleotides and/or the complementarity is at least about 80%, 85%, 90%, 95%, 96%, 97%, 98%, 99%, or 100%. In some embodiments, the PNA agent has a sequence that targets a non-promoter region of a gene. In some embodiments, the gene is a SARS-CoV-2 nucleic acid sequence, such as an RNA sequence of SARS-CoV-2 virus e.g., a positive or a negative RNA strand of the SARS-CoV-2 virus, or a nucleic acid sequence encoding a structural protein or an open reading frame (orf) protein. In some embodiments, the SARS-CoV-2 gene includes a variant or mutant sequence and the PNA agent has a sequence that targets a site including or consisting of the variant or mutant sequence (e.g., different strains of SARS-CoV-2).

In some embodiments, the PNA component incorporates a sense (mRNA) sequence of a target gene. In some embodiments, the PNA agent is targeted to a region of a gene encoding a SARS-CoV-2 structural protein, such as structural proteins S, N, M, or E. The sequences for these structural proteins are known in the art and publicly available as follows:

Spike (S): www.uniprot.org/uniprot/P0DTC2

Nucleoprotein (N): www.uniprot.org/uniprot/P0DTC9

Envelope (E): www.uniprot.org/uniprot/P0DTC4

Membrane (M): www.uniprot.org/uniprot/P0DTC5

One of skill in the art can deduce a nucleic acid sequence encoding one or more of the S, N, E, and M proteins of SARS-CoV-2 and design appropriate targeting nucleic acid sequences.

In some embodiments, the PNA agent has a sequence that targets a site in a gene, which PNA agent is characterized in that, when a system including a cell that expresses the gene is exposed to the PNA agent, expression of the gene is reduced by an amount within the range of 20% to 90% suppression of normal activity when the PNA agent is present as compared with otherwise comparable conditions when it is absent. In some embodiments, the PNA agent has a sequence that targets a site in a gene, which PNA agent is characterized in that, when a system including a cell that expresses the gene is exposed to the PNA agent, expression of the gene is reduced by an amount within the range of 20% to 90% when the PNA agent is present as compared with otherwise comparable conditions when it is absent. In some embodiments, protein product is reduced to less than 50% expression. In some embodiments, the cell is a human cell. In some embodiments, the system is or comprises an animal, such as a mouse, rate or non-human primate for example. In some embodiments, the system is or includes a primate. In some embodiments, the system is or includes a human. In some embodiments, the system is or includes a non-human primate. In some embodiments, the system is or includes a cell in culture.

In some embodiments, the PNA moiety has a length within the range of 13-18 nucleotides. In some embodiments, PNA moieties have palmitoyl lysine attached to the termini. In some embodiments, PNA moieties have palmitoyl lysine attached to both N- and C-termini. In some embodiments, PNA moieties have a delivery peptide length within the range of 8-12 amino acids. In some embodiments, PNA-peptide conjugates are intramolecularly stabilized by pi-interacting nucleoside bases. In some embodiments, the radius of gyration of the PNA agent is decreased within the range of 25% to 50%. In some embodiments, PNA agents, when contacted with a cell membrane, crosses the membrane 10 times as much as reference PNA agents lacking one or both terminal hydrophobic/cationic moieties. In some embodiments, gene suppression is approximately a magnitude more effective by employing cationic/hydrophobic Lys(palmitoyl)-Lys-Lys residues on both termini in comparison to a standard delivery peptide-PNA motif In a specific embodiment of the invention, the target gene is genomic RNA of the SARS-CoV-2 virus.

In some embodiments, a method for treating or reducing the risk of a disease, disorder, or condition including administering to a subject susceptible to the disease, disorder, or condition a PNA agent is provided. In some embodiments, the subject is suffering from or susceptible to SARS-CoV-2 infection. In some aspects, the PNA agent is provided to the subject prior to infection. In some aspects, the PNA agent is provided to the subject following infection by SARS-CoV-2.

In some embodiments, methods of reducing expression of a target gene in a cell including contacting a cell in which the target is expressed with at least one PNA agent; determining a level or activity of the target in the cell when the PNA agent is present as compared with a target reference level or activity observed under otherwise comparable conditions when it is absent; and classifying the at least one PNA agent as a target inhibitor if the level or activity of the target is significantly reduced when the PNA agent is present as compared with the target reference level or activity are provided.

In some embodiments, a method for identifying and/or characterizing PNA agents for target inhibition including contacting a system in which a target is expressed with at least one PNA agent; determining a level or activity of the target in the system when the PNA agent is present as compared with a target reference level or activity observed under otherwise comparable conditions when it is absent; and classifying the at least one PNA agent as a target inhibitor if the level or activity of the target is significantly reduced when the PNA agent is present as compared with the target reference level or activity is provided.

Any of the methods disclosed herein may include administering or using any of the PNA agents disclosed herein.

In some embodiments, the level or activity of the target includes a target mRNA level. In some embodiments, the level or activity of the target includes a target protein level. In some embodiments, the system includes an in vitro system. In some embodiments, the system includes an in vivo system. In some embodiments, the system is or includes cells.

In some embodiments, the level or activity of the target corresponds to cell viability. In some embodiments, a significant reduction in the level or activity of the target corresponds to a greater than 90% decrease in virus or virus-infected cell viability.

In some embodiments, the system is or includes tissue. In some embodiments, the system is or includes an organism. In some embodiments, the level or activity of the target corresponds to survival of the organism. In some embodiments, a significant reduction in the level or activity of the target includes a greater than 50% increase in survival of the organism.

In some embodiments, a significant reduction in the level or activity of the target comprises a greater than 30% reduction of target activity. In some embodiments, a significant reduction in the level or activity of the target includes a greater than 10%, 15%, 20%, 25%, 30%, 35%, 40%, 45%, 50%, 55%, 60%, 65%, 70%, 75%, 80%, 85%, 90%, 95%, 96%, 97%, 98%, 99% reduction of target levels. In some embodiments, a significant reduction in the level or activity of the target includes a greater than two-fold, three-fold, four-fold, five-fold, six-fold, seven-fold, eight-fold, nine-fold, ten-fold, fifteen-fold, twenty-fold, forty-fold, fifty-fold, sixty-fold, seventy-fold, eighty-fold, ninety-fold, one hundred-fold, two hundred-fold, three hundred-fold, four hundred-fold, five hundred-fold, six hundred-fold, seven hundred-fold, eight hundred-fold, nine hundred-fold, one thousand-fold, two thousand-fold, three thousand-fold, four thousand-fold, five thousand-fold, six thousand-fold, seven thousand-fold, eight thousand-fold, nine thousand-fold, ten thousand-fold or more. In some embodiments, the reference level is a historical reference. In some embodiments, the historical reference is recorded in a tangible and/or computer-readable medium.

In some embodiments, a pharmaceutical composition including the PNA agent described herein and pharmaceutically acceptable carrier is provided. In some embodiments, the pharmaceutical composition is formulated for direct administration into a target tissue. In some embodiments, the pharmaceutical composition is formulated for oral administration. In some embodiments, the pharmaceutical composition is formulated for parenteral administration, e.g., injection. In some embodiments, the pharmaceutical composition is formulated for intradermal administration. In some embodiments, the pharmaceutical composition is formulated for transdermal administration. In some embodiments, the pharmaceutical composition is formulated for administration by inhalation.

In some embodiments, the pharmaceutical composition is or includes a liquid. In some embodiments, the pharmaceutical composition is or includes a solid.

Additional features and advantages of the invention will be apparent from the following figures, definitions, detailed description and the claims.

### BRIEF DESCRIPTION OF THE DRAWINGS

FIGS. 1A-1E are graphs illustrating CPE inhibition by PNA agents in Vero E6 cells. FIG. 1A is a graph illustrating CPE inhibition by AB01971749. FIG. 1B is a graph illustrating CPE inhibition by AB01971744. FIG. 1C is a graph illustrating CPE inhibition by AB01971754. FIG. 1D is a graph illustrating CPE inhibition by AB01971748. FIG. 1E is a graph illustrating CPE inhibition by AB01971753.

FIGS. 2A-2E are graphs illustrating compound cytotoxicity by PNA agents in Vero E6 cells. FIG. 2A is a graph illustrating CPE inhibition by AB01971749. FIG. 2B is a graph illustrating CPE inhibition by AB01971744. FIG. 2C is a graph illustrating CPE inhibition by AB01971754. FIG. 2D is a graph illustrating CPE inhibition by AB01971748. FIG. 2E is a graph illustrating CPE inhibition by AB01971753.

### DETAILED DESCRIPTION OF THE INVENTION

The present invention is based, in part, upon the discovery that it is possible to produce peptide nucleic acid (PNA) agents that efficiently cross cell membranes and specifically target and suppress genes. Modifications to PNA agents improve the transmembrane permeability, target binding stability, solubility and specificity for target genes with specific sequences.

Those skilled in the art will recognize or be able to ascertain using no more than routine experimentation, many equivalents to the specific embodiments of the invention described herein. The scope of the present invention is not intended to be limited to the above Description. Likewise, those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

In the claims, articles such as “a”, “an” and “the” may mean one or more than one unless indicated to the contrary or otherwise evident from the context. Thus, for example, reference to “an antibody” includes a plurality of such antibodies, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are presenting, employed in, or otherwise relevant to a given product or process. Furthermore, it is to be understood that the invention encompasses all variations, combinations, and permutations in which one or more limitation, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for anyone of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understand of one of ordinary skill in the art, values that are expressed as ranges can assume any specific value or sub-range within the state ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or includes a natural product in that it is found in and/or is obtained from nature. In some embodiments, an agent is or includes one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, peptide nucleic acids, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Affinity: As is known in the art, “affinity” is a measure of the tightness with a particular ligand (e.g., an HA polypeptide) binds to its partner (e.g., an HA receptor). Affinities can be measured in different ways. In some embodiments, affinity is measured by a quantitative assay (e.g., glycan binding assays). In some such embodiments, binding partner concentration (e.g., HA receptor, glycan, etc.) may be fixed to be in excess of ligand (e.g., an HA polypeptide) concentration so as to mimic physiological conditions (e.g., viral HA binding to cell surface glycans). Alternatively, or additionally, in some embodiments, binding partner (e.g., HA receptor, glycan, etc.) concentration and/or ligand (e.g., an HA polypeptide) concentration may be varied. In some such embodiments, affinity (e.g., binding affinity) may be compared to a reference (e.g., a wild-type HA that mediates infection of a humans) under comparable conditions (e.g., concentrations).

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may include one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, of either sex, and at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, the animal is susceptible to infection by SARS-CoV-2. In some embodiments, an animal may be a transgenic animal, genetically engineered animal, and/or a clone.

Antagonist: As used herein, the term “antagonist” refers to an agent that i) inhibits, decreases or reduces the effects of another agent, for example that inactivates a nucleic acid; and/or ii) inhibits, decreases, reduces, or delays one or more biological events, for example, expression of one or more nucleic acids or stimulation of one or more biological pathways. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it exerts its influence directly upon the receptor) or indirect (in which case it exerts its influence by other than binding to the receptor; e.g., altering expression or translation of the receptor; altering signal transduction pathways that are directly activated by the receptor, altering expression, translation or activity of an agonist of the receptor).

Antibody polypeptide: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In some embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immunoglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In some embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In some embodiments, the “antibody polypeptide” is an antibody fragment that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively, or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity.

Antigen: An “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer other than a biologic polymer (e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or includes a recombinant antigen.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. In some embodiments, where a protein or polypeptide is biologically active, a portion of that protein or polypeptide that shares at least one biological activity of the protein or polypeptide is typically referred to as a “biologically active” portion.

Characteristic portion: As used herein, the term a “characteristic portion” of a substance, in the broadest sense, is one that shares some degree of sequence or structural identity with respect to the whole substance. In some embodiments, a characteristic portion shares at least one functional characteristic with the intact substance. For example, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance; epitope-binding specificity is one example. In some embodiments, a characteristic portion may be biologically active.

Combination therapy: The term “combination therapy”, as used herein, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity within a subject. For example, the present invention may include administration of a PNA agent and another antiviral agent, either prior to, simultaneously with, or following administration of the PNA agent.

Detection entity: The term “detection entity” as used herein refers to any element, molecule, functional group, compound, fragments thereof or moiety that facilitates detection of an agent (e.g., an antibody) to which it is joined. Examples of detection entities include, but are not limited to: various ligands, radionuclides (e.g., 3H, 14C, 18F, 19F, 32P, 35S, 135I, 125I, 123I, 64Cu, 187Re, 111In, 90Y, 99mTc, 177Lu, 89Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Diagnostic information: As used herein, diagnostic information or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as a viral infection or cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosage form: As used herein, the terms “dosage form” and “unit dosage form” refer to a physically discrete unit of a therapeutic composition to be administered to a subject. Each unit contains a predetermined quantity of active material (e.g., a therapeutic agent). In some embodiments, the predetermined quantity is one that has been correlated with a desired therapeutic effect when administered as a dose in a dosing regimen. Those of ordinary skill in the art appreciate that the total amount of a therapeutic composition or agent administered to a particular subject is determined by one or more attending physicians and may involve administration of multiple dosage forms.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, a dosing regimen is or has been correlated with a desired therapeutic outcome, when administered across a population of patients.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, a “functional” biological molecule is a biological molecule in a form in which it exhibits a property and/or activity by which it is characterized. A biological molecule may have two functions (i.e., bifunctional) or many functions (i.e., multifunctional).

Gene: As used herein, the term “gene” has its meaning as understood in the art. In some embodiments, the term “gene” may include gene regulatory sequences (e.g., promoters, enhancers, etc.) and/or intron sequences. In some embodiments, the term refers to nucleic acids that do not encode proteins but rather encode functional RNA molecules such as tRNAs, RNAi-inducing agents, etc. Alternatively, or additionally, in some embodiments, the term “gene”, as used in the present application, refers to a portion of a nucleic acid that encodes a protein. Whether the term encompasses other sequences (e.g., non-coding sequences, regulatory sequences, etc.) will be clear from context to those of ordinary skill in the art.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Lysine or lysine residue: As used herein, the term “lysine” or “lysine residue” refers to the basic amino acid residue and its derivatives. Such derivatives include those lysine residues with side chain modifications. Lysine derivatives include e-palmitoyl lysine or Lys(palmitoyl-(dLys)2. Marker: A marker, as used herein, refers to an agent whose presence or level is a characteristic of a particular tumor or metastatic disease thereof. For example, in some embodiments, the term refers to a gene expression product that is characteristic of a particular tumor, tumor subclass, stage of tumor, etc. Alternatively, or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

SARS-CoV-2 gene: As used herein, the term “SARS-CoV-2 gene” refers to those genes whose products are produced by the coronavirus SARS-CoV-2, including but not limited to the S, M, N and E genes and gene products.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes infection by or at risk of infection by SARS-CoV-2.

Peptide: The term “peptide” refers to two or more amino acids joined to each other by peptide bonds or modified peptide bonds. In some embodiments, “peptide” refers to a polypeptide having a length of less than about 100 amino acids, less than about 50 amino acids, less than 20 amino acids, or less than 10 amino acids.

Peptide nucleic acid: The term “peptide nucleic acid” refers to synthetic polymers similar to DNA or RNA, but lacking deoxyribose and ribose sugar backbones, respectively. Peptide nucleic acids possess a backbone composed of repeating N-(2-aminoethyl)-glycine units linked by peptide bonds. Purine and pyrimidine bases are linked to the backbone by a methylene bridge and carbonyl group.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Prognostic and predictive information: As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Promoter: As used herein, the term “promoter” refers to regions of DNA that serve as initiation sites for transcription of a particular gene. Promoter sequences are often open/unraveled and await binding to other elements.

Protein: As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or includes a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may include natural amino acids, non-natural amino acids, synthetic amino acids, and/or combinations thereof. In some embodiments, proteins are or include antibodies, antibody polypeptides, antibody fragments, biologically active portions thereof, and/or characteristic portions thereof.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a virus infection response. Virus, virus-infected cell or a subject response may be measured according to a wide variety of criteria, including clinical criteria and objective criteria. Techniques for assessing response include, but are not limited to, clinical examination, positron emission tomography, chest X-ray CT scan, Mill, ultrasound, endoscopy, laparoscopy, presence or level of viral load or anti-viral antibodies in a sample obtained from a subject, cytology, and/or histology. One of ordinary skill in the art will be able to select appropriate criteria.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification of nucleic acid etc. Typically, the present invention includes blood, plasma, saliva or nasal swab samples.

Specific binding: As used herein, the terms “specific binding” or “specific for” or “specific to” refer to an interaction (typically non-covalent) between a target entity (e.g., a target protein or polypeptide) and a binding agent (e.g., an antibody, such as a provided antibody). As will be understood by those of ordinary skill, an interaction is considered to be “specific” if it is favored in the presence of alternative interactions. In some embodiments, an interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc.). Specificity may be evaluated in the context of additional factors such as the affinity of the binding molecule for the target molecule versus the affinity of the binding molecule for other targets (e.g., competitors). If a binding molecule exhibits a high affinity for a target molecule that it is desired to detect and low affinity for non-target molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (cancer, viral or other pathogen infection) has been diagnosed with and/or exhibits one or more symptoms of the disease, disorder, or condition.

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many COVID-19 patients have no symptoms and are asymptomatic. It is not intended that the present invention be limited only to cases where the symptoms are eliminated. The present invention specifically contemplates treatment such that one or more symptoms is/are reduced (and the condition of the subject is thereby “improved”), albeit not completely eliminated, e.g., reduction in viral load.

Therapeutic agent: As used herein, the phrase “therapeutic agent” refers to any agent that has a therapeutic effect and/or elicits a desired biological and/or pharmacological effect, when administered to a subject.

Therapeutically effective amount: As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., viral infection, cancer). Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The terms PNA and PNA moiety are used interchangeably herein. The terms PNA agent and PNA derivative are used interchangeably herein.

In some embodiments, PNA agents including a PNA-moiety; and a first cationic and/or hydrophobic moiety at a first end of the PNA moiety; and a second cationic and/or hydrophobic moiety at a second end of the PNA moiety are provided. In some embodiments, PNA agents wherein the first and/or second cationic and/or hydrophobic moiety is or includes a peptide are provided. In some embodiments, the first and/or second cationic and/or hydrophobic peptide includes or consists of lysine residues. In some embodiments, at least one lysine residue includes a palmitoyl side chain moiety. In some embodiments, the first and/or second cationic and/or hydrophobic peptide includes amines. In some embodiments, PNA agents have a sequence that does not form hairpin loops. In some embodiments, PNA agents should have a sequence that does not form hairpin loops. In some embodiments, PNA agents should have a sequence that contains less than 60% purines.

In some embodiments, the first and/or second cationic and/or hydrophobic moiety is a targeting and delivery moiety that aids delivery of PNA through the cell membrane whereas the cationic moiety guides stability of the PNA against the target phosphate backbone. In some embodiments, the second cationic and/or hydrophobic moiety is or includes a second cationic peptide which functions in the same capacity as the first cationic/hydrophobic moiety. In some embodiments, the cationic and/or hydrophobic moiety is at the PNA agent's N-terminus. In some embodiments, the targeting, or hydrophobic and/or cationic moiety is at the PNA agent's C-terminus. In some embodiments, the first cationic and/or hydrophobic moiety is a targeting moiety that aids delivery of PNA through the cell membrane and is attached at the PNA agent's N-terminus; and the second cationic and/or hydrophobic moiety is or includes a cationic peptide that is attached at the PNA agent's C-terminus and guides stability of the PNA against the target phosphate backbone. In some embodiments, a second cationic peptide N-terminal to the PNA peptide and C-terminal to the targeting moiety is provided.

In some embodiments, PNA agents having a sequence that targets a SARS-CoV-2 nucleic acid sequence are provided. In some embodiments, PNA agents have a sequence that targets a 13-20 nucleotide sequence of a gene with 75% or greater complementarity. In some embodiments, PNA agents have a sequence that targets a nucleic acid whose length is at least 14, 15, 16, 17, or 18 nucleotides and/or the complementarity is at least about 80%, 85%, 90%, 95%, 96%, 97%, 98%, 99%, or 100%.

In some embodiments, the PNA component incorporates a sense (mRNA) sequence of a target gene.

In some embodiments, PNA agents have a sequence that targets a non-promoter region of a gene. In some embodiments, the gene is a SARS-CoV-2 gene. In some embodiments, SARS-CoV-2 genes include a mutant or variant sequence element and PNA agents have a sequence that targets a site including or consisting of the mutant sequence element. For example, several strains of SARS-CoV-2 have now been identified, each having variations in particular regions of their nucleic acid sequence.

In some embodiments, PNA agents have a sequence that targets a site in a gene, which PNA agents are characterized in that, when a system including a cell that expresses the gene is exposed to the PNA agent, expression of the gene is reduced by an amount within the range of 50% to 100% when the PNA agent is present as compared with otherwise comparable conditions when it is absent. In some embodiments, the ideal range is dependent upon the response measured by decreased cell proliferation. In some embodiments, the cell is a human cell. In some embodiments, the system is or includes an animal. In some embodiments, the system is or includes a primate. In some embodiments, the system is or includes a human. In some embodiments, the system is or includes the cell in culture.

In some embodiments, PNA moieties have a length within the range of 13-18 nucleotides. In some embodiments, PNA moieties have palmitoyl lysine attached to the termini. In some embodiments, PNA moieties have palmitoyl lysine attached to both N- and C-termini. In some embodiments, PNA moieties have a delivery peptide length within the range of 8-12 amino acids. In some embodiments, PNA-peptide conjugates are intramolecularly stabilized by pi-interacting nucleoside bases. In some embodiments, the radius of gyration of the PNA agent is decreased within the range of 25% to 50% by hydrophobic solvent exclusion driving the N- and C-terminal palmitoyl lysines proximal to each other. In some embodiments, PNA agents, when contacted with a cell membrane, crosses the membrane 10 times more quickly in comparison to reference PNA agents lacking one or both terminal hydrophobic/cationic moieties. In some embodiments, PNA agents cross membranes a magnitude more easily based upon results from cell proliferation experiments comparing with and without terminal palmitoyl lysines.

In some embodiments, a PNA moiety has a first cationic moiety and a first hydrophobic moiety at a first end, and a second cationic moiety and a second hydrophobic moiety at a second end, and the first cationic moiety and the first hydrophobic moiety at the first end are part of one amino acid, and the second cationic moiety and the second hydrophobic moiety at the second end are part of one amino acid.

In some embodiments, methods for treating or reducing the risk of a disease, disorder, or condition including administering to a subject susceptible to the disease, disorder, or condition PNA agents are provided. In some embodiments, subjects suffering from or susceptible to viral infection by SARS-CoV-2 are provided.

In some embodiments, methods of reducing expression of a target gene in a cell including contacting a cell in which the target is expressed with at least one PNA agent; determining a level or activity of the target in the cell when the PNA agent is present as compared with a target reference level or activity observed under otherwise comparable conditions when it is absent; and classifying the at least one PNA agent as a target inhibitor if the level or activity of the target is significantly reduced when the PNA agent is present as compared with the target reference level or activity are provided.

In some embodiments, methods for identifying and/or characterizing PNA agents for target inhibition including contacting a system in which a target is expressed with at least one PNA agent; determining a level or activity of the target in the system when the PNA agent is present as compared with a target reference level or activity observed under otherwise comparable conditions when it is absent; and classifying the at least one PNA agent as a target inhibitor if the level or activity of the target is significantly reduced when the PNA agent is present as compared with the target reference level or activity are provided. In some embodiments, suppressing a SARS-CoV-2 gene is measured by determining the activity level by the amount of suppression of cell proliferation; the suppression of SARS-CoV-2 gene mRNA; and the suppression of SARS-CoV-2 gene protein product. PNAs without any terminal (d)lysine-(d)lysine palmitoyl lysine, with one terminal (d)lysine-(d)lysine-palmitoyl lysine, and with both terminal (d)lysine-(d)lysine-palmitoyl lysine have been evaluated. In some embodiments, PNAs conjugated to NLS, TAT, or any other delivery peptide, incorporating both terminal (d)lysine-(d)lysine-palmitoyl lysine show significantly better gene suppression than those with only a single terminus derivatized or no termini derivatized.

In some embodiments, one or more of the following PNA agents may be used in connection with the present invention: PNAs with terminal peptides including tetra-substituted ammonium or tri-substituted sulfonium moieties.

In some embodiments, the level or activity of the target includes a target mRNA level. In some embodiments, the level or activity of the target includes a target protein level. In some embodiments, the level or activity of the target corresponds to cell viability. In some embodiments, a significant reduction in the level or activity of the target corresponds to a greater than 50% increase in cell viability. In some embodiments, complete suppression of gene expression is not necessary for significant suppression of cell proliferation/decreasing cell viability.

In some embodiments, the system includes an in vitro system. In some embodiments, the system includes an in vivo system. In some embodiments, the system is or includes cells.

In some embodiments, the system is or includes tissue. In some embodiments, the system is or includes an organism. In some embodiments, the level or activity of the target corresponds to survival of the organism. In some embodiments, a significant reduction in the level or activity of the target includes a greater than 50% increase in survival of the organism.

In some embodiments, a significant reduction in the level or activity of the target includes a greater than 30-50% reduction of target activity. In some embodiments, a significant reduction in the level or activity of the target includes a greater than 50-100% reduction of target activity. In some embodiments, a significant reduction in the level or activity of the target includes a greater than 50% reduction of target levels. In some embodiments, reduction of gene target expression by 30-50% significantly reduces cell proliferation or virus levels in cell culture. Exemplary assays for determining virus levels and virus replication are shown in Rumlova and Ruml, Biotechnology Advances 36 (218) 557-576, which is herein incorporated by reference in its entirety, and the Examples herein.

In some embodiments, the reference level is a historical reference. In some embodiments, the historical reference is recorded in a tangible and/or computer-readable medium.

In some embodiments, a pharmaceutical composition including PNA agents and pharmaceutically acceptable carriers are provided. In some embodiments, pharmaceutical composition is formulated for direct administration into a target tissue. In some embodiments, the pharmaceutical composition is formulated for oral administration. In some embodiments, the pharmaceutical composition is formulated for parenteral administration. In some embodiments, the pharmaceutical composition is formulated for intradermal administration. In some embodiments, the pharmaceutical composition is formulated for transdermal administration. In some embodiments, the pharmaceutical composition is formulated for administration by inhalation. In some embodiments, the pharmaceutical composition is or includes a liquid. In some embodiments, the pharmaceutical composition is or includes a solid.

Peptide Nucleic Acid (PNA) Agent Structure

Peptide nucleic acids are synthetic polymers with similarities to DNA and RNA. PNAs possess backbones of repeating N-(2-aminoethyl)-glycine units that are linked by peptide bonds. PNAs are also called PNA moieties herein. This differs from backbones of DNA and RNA which are composed of deoxyribose and ribose sugar backbones, respectively. Furthermore, pyrimidine and purine bases are linked to the PNA backbone by carbonyl groups and methylene bridges. PNA backbones contain no charged phosphate groups. Therefore, due to a lack of electrostatic repulsion, binding between PNA sequences and DNA (or RNA) strands is stronger than binding between two DNA (or RNA) strands. Because of the higher binding strength, PNA oligomers longer than 20-25 bases are usually not necessary. Increasing the length of PNA strands could reduce specificity for target DNA (or RNA) sequences. A PNA/DNA mismatch has greater instability than a DNA/DNA mismatch; PNAs exhibit greater specificity than DNA when binding to complementary sequences. The lack of charged phosphate groups also contributes to the hydrophobic nature of PNAs, which cannot cross cellular membranes without some modification. These modifications can include, but are not limited to, covalently coupling a cell penetrating peptide and/or adding cationic/hydrophobic peptides. PNAs are also stable over a wide pH range and are resistant to enzyme degradation as they are not recognized by either proteases or nucleases.

In some embodiments, PNA agents are complementary to a target sequence. In some embodiments, they are exact copies of a mRNA sequence expressed by a gene of interest. In some embodiments, this is also the sense strand sequence of the gene. In some embodiments, PNA agents can be created complementary to any gene of interest, e.g., the S gene of SARS-CoV-2.

Embodiments of the present invention are drawn to methods of improving the ability of PNA agents to cross cellular membranes. In some embodiments, the physico-chemical properties of the PNA agents have been modified to improve delivery across cell membranes by adding cationic/hydrophobic delivery peptides. In some embodiments, the hydrophobic and cationic terminal peptides together facilitate passive transport across membranes. PNA agents comprised of hydrophobic e-palmitoyl lysines at the termini have improved capabilities for crossing cellular membranes compared to standard PNA-peptide conjugates. In some embodiments, the terminal hydrophobic moieties in this design also allow the termini to be hydrophobically driven together decreasing the radius of gyration of the polymer. The smaller size allows for better transport. In some embodiments, having both ends of the PNA polymer derivatized more thoroughly imparts delivery functionalization of this large molecule. Hydrophobic e-palmitoyl lysine termini are driven together by solvent exclusion and the PNA-peptide conjugate is intramolecularly further stabilized by pi-interacting nucleoside bases. The PNA-peptide conjugate becomes more compact due to a decreased radius of gyration, thus allowing it to more easily permeate lipid bilayers. In some embodiments, the PNA agent is comprised of Lys(palmitoyl)-(dLys)2 at the N- and C-termini, bracketing a delivery peptide of ^{˜}10 amino acids in length and a ^{˜}15-18mer PNA. In some embodiments, the typical structure of PNA agents include: a delivery peptide of approximately 10 amino acids in length and a ^{˜}15-18mer PNA located within the bounds of two Lys(palmitoyl)-(dLys)2-termini attached by d-lysine. For example:

Lys(palmitoyl)-(dLys)2-delivery peptide-PNA-(dLys)2-Lys(palmitoyl)

In some embodiments, PNA agents employ a modified NLS delivery peptide. In some embodiments, PNA agents employ a modified TAT delivery peptide.

In some embodiments, PNA agents used against a BRAF V600E target include:

AcNH-Lys(palmitoyl)-dLys-dLys-CCTCAAGAGTAATAATAT-dLys-dPro-dLys-dLys-dLys-dArg-dLys-dVal-dLys-dLys-Lys(palmitoyl)-CONH2 [I-292-3 L2LP (employing a NLS delivery peptide)] and AcNH-Lys(palmitoyl)-dLys-dLys-CCTCAAGAGTAATAATAT-dLys-dArg3-dGln-dArg2-dLys2-dArg-Gly-dTyr-dLys-dLys-Lys(palmitoyl)-CONH2. [I-292-9 L2 (employing a modified TAT delivery peptide)]

Nucleic Acid Targeting

In some embodiments, the cationically charged termini improve the ability of the PNA to target specific nucleic acid sequences. Stabilizing the cationically charged lysine-derivatized termini against the anionic DNA offers a kinetically faster binding by terminal nucleation as per the Zimm-Bragg statistical model. This enables the PNA to target non-promoter sequences in an improved manner, which is especially unexpected given that promoter sequences are usually open/unraveled and awaiting binding while non-promoter regions of genes are less accessible. PNA is stabilized against its DNA target merely for lacking repulsive anionic phosphate-phosphate repulsive forces (enthalpic advantage). The cationic ends of the PNA-peptide improve the entropic component of binding by stabilizing the more configurationally free termini of the PNA-peptide against the target.

In some embodiments, the PNA of the PNA-peptide conjugate is of standard design—the length range is usually from 13-18 bases. For lengths less than 13 bases the binding becomes much less thermodynamically favorable due to decreased enthalpy of binding. For lengths greater than 18 bases do not offer more of a thermodynamic advantage as the gain in enthalpic binding energy is offset by the kinetic disadvantage of properly positioning such a long strand (more intramolecular substrates could compete with the binding state),In some embodiments, the PNA moiety has a sequence that targets an RNA sequence. The PNA agents described herein include a PNA moiety with a sequence that targets a SARS-CoV-2 gene.

Coronaviruses are spherical enveloped viruses containing a single strand of positive-sense RNA, similar to host mRNA. Following receptor binding, the virus must next gain access to the host cell cytosol. This is generally accomplished by acid-dependent proteolytic cleavage of S protein by a cathepsin, TMPRRS2 or another protease, followed by fusion of the viral and cellular membranes, ultimately resulting in release of the viral genome into the cytoplasm. The next step in the coronavirus lifecycle is the translation of the replicase gene from the virion genomic RNA. Since coronaviruses have a single positive stranded RNA genome, they can directly produce their proteins and new genomes in the host cell cytoplasm. The first step is for the virus to synthesize its RNA polymerase, which only recognizes and produce viral RNA; this enzyme produces negative stranded RNA, using the positive strand as a template. The negative strands then serve as (1) templates to transcribe small subgenomic positive RNAs that are used to synthesize all other viral proteins, and (2) templates for the replication of new positive stranded RNA genomes. Newly replicated positives stranded RNA genomes bind to newly synthesized nucleocapsid (N) proteins and newly synthesized membrane (M) proteins are integrated into the host cell endoplasmic reticulum membrane, along with newly synthesized spike (S) and envelope (E) proteins. After binding, assembled nucleocapsids with helical twisted RNA bud into the endoplasmic reticulum lumen and are encased with its membraned. The newly formed virions are then transported to the cell membraned by Golgi vesicles, and exocytosed into the extracellular space.

As used herein, a PNA moiety having a sequence that targets “an RNA sequence” refers to a PNA moiety that targets a SARS-CoV-2 virus RNA molecule regardless of its type of strand, that is the PNA agents described herein can target SARS-CoV-2 virus RNA at any stage in the replication cycle of the virus. Therefore, the PNA agents described herein have a sequence that can target a positive RNA strand or a negative RNA strand of a SARS-CoV-2 virus.

In some aspects, the PNA moiety has a sequence including any one of SEQ ID NOs: 1-5. In other aspects, the PNA moiety has a sequence that targets an RNA sequence including any one of SEQ ID NOs: 6-10.

In some embodiments, the PNA agents target specific genes or genetic sequences. PNA agents can be designed to target genes possessing known mutated sequences as well as sites of genetic translocations. In some embodiments, the PNA agents target SARS-CoV-2 genes. In some embodiments, the PNA agents can target mutant or variant SARS-CoV-2 genes.

In one aspect, the PNA agents target a SARS-CoV-2 nucleic acid sequence encoding a structural protein or an open reading frame (orf) protein. In another aspect, the SARS-CoV-2 nucleic acid sequence encodes a structural protein selected from a Spike (S) protein, Nucleocapsid (N) protein, Envelope (E) protein, or Membrane (M) protein. In some aspects, the PNA agent targets a nucleic acid sequence comprising the S protein.

Applications of PNA Agents

Targeting and binding by PNA agents would have uses as research tools, medical diagnostics and pharmaceutical treatments. In some embodiments, PNA agents can be used to target and bind specific genetic sequences. In some embodiments, PNA agents can be used to suppress expression of genetic sequences. PNA agents targeted to specific genes can serve as valuable research tools in understanding the function of those genes. Suppressing the expression of particular gene products would help elucidate and discover the role of those products in different biological pathways.

PNA agents can target and bind to mutated or variant genetic sequences and suppress the expression of mutant SARS-CoV-2 genes, thereby suppressing and/or treating the viral infection. In some embodiments, PNA agents are used to treat SARS-CoV-2 infection due to any of the strains of the virus.

Pharmaceutical Compositions

The present invention also provides compositions including one or more provided PNA agents. In some embodiments, the present invention provides at least one PNA-conjugate and at least one pharmaceutically acceptable excipient. Such pharmaceutical compositions may optionally comprise and/or be administered in combination with one or more additional therapeutically or biologically active substances. In some embodiments, provided pharmaceutical compositions are useful in medicine or the manufacture of medicaments. In some embodiments, provided pharmaceutical compositions are useful as prophylactic agents (i.e., vaccines) in the treatment or prevention of pathogen infection, e.g., virus and respiratory infections associated therewith, cancer and neurodegenerative disorders. In some embodiments, provided pharmaceutical compositions are useful in therapeutic applications, for example in individuals suffering from a disease; e.g., as delivery vehicles capable of specifically targeting cytotoxic agents or compounds that block aberrant cellular signaling. In some embodiments, the pharmaceutical compositions are simultaneously useful in diagnostic applications and therapeutic applications. In some embodiments, pharmaceutical compositions are formulated for administration to humans. In some embodiments, the pharmaceutical compositions include a PNA agent in combination with or conjugated to a therapeutic agent or other therapeutic as defined herein.

For example, pharmaceutical compositions may be provided in a sterile injectable form (e.g., a form that is suitable for subcutaneous injection or intravenous infusion). In some embodiments, pharmaceutical compositions are provided in a liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions include one or more pharmaceutically acceptable excipients (e.g., preservative, inert diluent, dispersing agent, surface active agent and/or emulsifier, buffering agent, etc.). In some embodiments, pharmaceutical compositions include one or more preservatives. In some embodiments, pharmaceutical compositions include no preservatives.

In some embodiments, pharmaceutical compositions are provided in a form that can be refrigerated and/or frozen. In some embodiments, pharmaceutical compositions are provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of PNA compositions for longer than the specified time results in PNA degradation.

Liquid dosage forms and/or reconstituted solutions may include particulate matter and/or discoloration prior to administration. In some embodiments, a solution should not be used if discolored or cloudy and/or if particulate matter remains after filtration.

Pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient; for example, a peptide nucleic acid agent. The amount of the active ingredient is generally equal to a dose that would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

Pharmaceutical compositions of the present invention may additionally include a pharmaceutically acceptable excipient, which, as used herein, may be or include solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

Conjugates Generally

Multifunctional agents described herein include multiple entities, each having at least one function. Certain embodiments of contemplated multifunctional agents include a targeting entity and at least one of the following entities: a detection entity, a therapeutic entity, and a diagnostic entity. In some embodiments, a multifunctional agent of the invention contains a targeting entity, a therapeutic entity and a detection entity. In some embodiments, the entities of an agent may be conjugated to one another. Conjugation of various entities to form a multifunctional agent is not limited to particular modes of conjugation. For example, two entities may be covalently conjugated directly to each other. Alternatively, two entities may be indirectly conjugated to each other, such as via a linker entity. In some embodiments, a multifunctional agent may include different types of conjugation within the agent, such that some entities of the agent are conjugated via direct conjugation while other entities of the agent are indirectly conjugated via one or more linkers. In some embodiments, a multifunctional agent of the invention includes a single type of a linker entity. In some embodiments, a multifunctional agent of the invention includes more than one type of linker entities. In some embodiments, a multifunctional agent includes a single type of linker entities but of varying length.

In some embodiments, there is a covalent association between or among entities contained in a multifunctional agent. As will be appreciated by one skilled in the art, the moieties may be attached to each other either directly or indirectly (e.g., through a linker, as described below).

In some embodiments, where one entity (such as a targeting entity) and a second entity of a multifunctional agent are directly covalently linked to each other, such direct covalent conjugation can be through a linkage (e.g., a linker or linking entity) such as an amide, ester, carbon-carbon, disulfide, carbamate, ether, thioether, urea, thiourea, isothiourea, amine, or carbonate linkage. Covalent conjugation can be achieved by taking advantage of functional groups present on the first entity and/or the second entity of the multifunctional agent. Alternatively, a non-critical amino acid may be replaced by another amino acid that will introduce a useful group (such as amino, carboxy or sulfhydryl) for coupling purposes. Alternatively, an additional amino acid may be added to at least one of the entities of the multifunctional agent to introduce a useful group (such as amino, carboxy or sulfhydryl) for coupling purposes. Suitable functional groups that can be used to attach moieties together include, but are not limited to, amines, anhydrides, hydroxyl groups, carboxy groups, thiols, and the like. An activating agent, such as a carbodiimide, can be used to form a direct linkage. A wide variety of activating agents are known in the art and are suitable for conjugating one entity to a second entity.

In some embodiments, entities of a multifunctional agent embraced by the present invention are indirectly covalently linked to each other via a linker group. Such a linker group may also be referred to as a linker or a linking entity. This can be accomplished by using any number of stable bifunctional agents well known in the art, including homofunctional and heterofunctional agents (for examples of such agents, see, e.g., Pierce Catalog and Handbook). The use of a bifunctional linker differs from the use of an activating agent in that the former results in a linking moiety being present in the resulting conjugate (agent), whereas the latter results in a direct coupling between the two moieties involved in the reaction. The role of a bifunctional linker may be to allow reaction between two otherwise inert moieties. Alternatively or additionally, the bifunctional linker that becomes part of the reaction product may be selected such that it confers some degree of conformational flexibility to the agent (e.g., the bifunctional linker comprises a straight alkyl chain containing several atoms, for example, the straight alkyl chain contains between 2 and 10 carbon atoms). Alternatively or additionally, the bifunctional linker may be selected such that the linkage formed between a provided antibody and therapeutic agent is cleavable, e.g., hydrolysable (for examples of such linkers, see e.g. U.S. Pat. Nos. 5,773,001; 5,739,116 and 5,877,296, each of which is incorporated herein by reference in its entirety). Such linkers, for example, may be used when higher activity of certain entities, such as a targeting agent and/or of a therapeutic entity is observed after hydrolysis of the conjugate. Exemplary mechanisms by which an entity may be cleaved from a multifunctional agent include hydrolysis in the acidic pH of the lysosomes (hydrazones, acetals, and cis-aconitate-like amides), peptide cleavage by lysosomal enzymes (the capthepsins and other lysosomal enzymes), and reduction of disulfides). Another mechanism by which such an entity is cleaved from the multifunctional agent includes hydrolysis at physiological pH extra- or intra-cellularly. This mechanism applies when the crosslinker used to couple one entity to another entity is a biodegradable/bioerodible component, such as polydextran and the like.

For example, hydrazone-containing multifunctional agents can be made with introduced carbonyl groups that provide the desired release properties. Multifunctional agents can also be made with a linker that includes an alkyl chain with a disulfide group at one end and a hydrazine derivative at the other end. Linkers containing functional groups other than hydrazones also have the potential to be cleaved in the acidic milieu of lysosomes. For example, multifunctional agents can be made from thiol-reactive linkers that contain a group other than a hydrazone that is cleavable intracellularly, such as esters, amides, and acetals/ketals.

Another example of class of pH sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used.

Another potential release method for conjugates of the therapeutic agents is the enzymatic hydrolysis of peptides by the lysosomal enzymes. In one example, a provided antibody is attached via an amide bond to para-aminobenzyl alcohol and then a carbamate or carbonate is made between the benzyl alcohol and the therapeutic agent. Cleavage of the peptide leads to collapse of the amino benzyl carbamate or carbonate, and release of the therapeutic agent. In another example, a phenol can be cleaved by collapse of the linker instead of the carbamate. In another variation, disulfide reduction is used to initiate the collapse of a para-mercaptobenzyl carbamate or carbonate.

Useful linkers which may be used as a linking entity of a multifunctional agent provided herein include, without limitation: polyethylene glycol, a copolymer of ethylene glycol, a polypropylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid, a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group.

Some embodiments of the invention utilize multifunctional agents that include at least one non-covalently associated entity. Examples of non-covalent interactions include, but are not limited to, hydrophobic interactions, electrostatic interactions, dipole interactions, van der Waals interactions, and hydrogen bonding. Irrespective of the nature of the binding, interaction, or coupling, the association between a first entity and a second entity is, in some embodiments, selective, specific and strong enough so that the second entity contained in the agent does not dissociate from the first entity before or during transport/delivery to and into the target. Thus, association among multiple entities of a multifunctional agent may be achieved using any chemical, biochemical, enzymatic, or genetic coupling known to one skilled in the art.

Therapeutic Conjugates

As described herein, PNA agents may include part of multifunctional agents with therapeutic utility related to viral infection. Examples of therapeutic utilities in the context of the present disclosure include, without limitation, utility associated with targeting (e.g., binding specific gene sequences), utility associated with therapeutic effects (e.g., cytotoxic and/or cytostatic effects, anti-proliferative effects, anti-angiogenic effects, reducing symptoms etc.), and utility associated with diagnosis, detection or labeling, etc.

A targeting entity is a molecular structure that can be contained in an agent which affects or controls the site of action by specifically interacting with, or has affinity for, a target of interest. As an example, a target may be a molecule or molecular complex present on a cell surface, e.g., certain cell types, tissues, etc. In some embodiments of the invention, the target is virus-associated, and the targeting entity is a PNA agent. Use of targeting moieties for agents, such as therapeutic agents, is known in the art.

In some embodiments, the PNA agents are multifunctional agents including a gene targeting entity, which essentially consists of a PNA agent, conjugated to one or more therapeutic agents, e.g., an anti-viral agent, for example Remdesivir. Non-limiting embodiments of useful conjugates of PNA agents that may be used in the diagnosis or assessment of, treatment of and the manufacture of medicaments for viral infections or other disorders are provided below.

PNA agents may have any of a variety of uses including, for example, use as anti-viral or other therapeutic agents, probes, primers, etc. Nucleic acid agents may have enzymatic activity (e.g., ribozyme activity), gene expression inhibitory activity (e.g., as antisense or siRNA agents, etc.), and/or other activities. Nucleic acids agents may be active themselves or may be vectors that deliver active nucleic acid agents (e.g., through replication and/or transcription of a delivered nucleic acid). For purposes of the present specification, such vector nucleic acids are considered “therapeutic agents” if they encode or otherwise deliver a therapeutically active agent, even if they do not themselves have therapeutic activity.

In some embodiments, conjugates of PNA agents include a nucleic acid therapeutic agent that is a ribozyme. As used herein, the term “ribozyme” refers to a catalytic RNA molecule that can cleave other RNA or DNA molecules in a target-specific manner. Ribozymes can be used to downregulate the expression of any undesirable products of genes of interest. Examples of ribozymes that can be used in the practice of the present invention include, but are not limited to, those specific for SARS-CoV-2 gene RNA.

In some embodiments, entities or moieties within conjugates of the PNA agents include a photosensitizer used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer typically is in a form suitable for administration, and also in a form that can readily undergo cellular internalization at the target site, often with some degree of selectivity over normal tissues.

Conjugates of PNA agents associated with a photosensitizer can be used as new delivery systems in PDT. In addition to reducing photosensitizer aggregation, delivery of photosensitizers according to the present invention exhibits other advantages such as increased specificity for target tissues/organ and cellular internalization of the photosensitizer.

Photosensitizers suitable for use in the present invention include any of a variety of synthetic and naturally occurring molecules that have photosensitizing properties useful in PDT. In some embodiments, the absorption spectrum of the photosensitizer is in the visible range, typically between 350 nm and 1200 nm, preferably between 400 nm and 900 nm, e.g., between 600 nm and 900 nm. Suitable photosensitizers that can be coupled to toxins according to the present invention include, but are not limited to, porphyrins and porphyrin derivatives (e.g., chlorins, bacteriochlorins, isobacteriochlorins, phthalocyanines, and naphthalocyanines); metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid (R. W. Redmond and J. N. Gamlin, Photochem. Photobiol., 1999, 70: 391-475).

Exemplary photosensitizers suitable for use in the present invention include those described in U.S. Pat. Nos. 5,171,741; 5,171,749; 5,173,504; 5,308,608; 5,405,957; 5,512,675; 5,726,304; 5,831,088; 5,929,105; and 5,880,145 (the contents of each of which are incorporated herein by reference in their entirety).

In some embodiments, conjugates of PNA agents include a radiosensitizer. As used herein, the term “radiosensitizer” refers to a molecule, compound or agent that makes tumor cells more sensitive to radiation therapy. Administration of a radiosensitizer to a patient receiving radiation therapy generally results in enhancement of the effects of radiation therapy. The advantage of coupling a radiosensitizer to a targeting entity (e.g., PNA agents capable of targeting intratumoral genetic sequences) is that the radiosensitize effects only on target cells. For ease of use, a radiosensitizer should also be able to find target cells even if it is administered systemically. However, currently available radiosensitizers are typically not selective for tumors, and they are distributed by diffusion in a mammalian body. PNA agents conjugates of the present invention can be used as a new delivery system for radiosensitizers.

In some embodiments, conjugates of the PNA agents may be used in directed enzyme prodrug therapy. In a directed enzyme prodrug therapy approach, a directed/targeted enzyme and a prodrug are administered to a subject, wherein the targeted enzyme is specifically localized to a portion of the subject's body where it converts the prodrug into an active drug. The prodrug can be converted to an active drug in one step (by the targeted enzyme) or in more than one step. For example, the prodrug can be converted to a precursor of an active drug by the targeted enzyme. The precursor can then be converted into the active drug by, for example, the catalytic activity of one or more additional targeted enzymes, one or more non-targeted enzymes administered to the subject, one or more enzymes naturally present in the subject or at the target site in the subject (e.g., a protease, phosphatase, kinase or polymerase), by an agent that is administered to the subject, and/or by a chemical process that is not enzymatically catalyzed (e.g., oxidation, hydrolysis, isomerization, epimerization, etc.).

Some embodiments of the invention utilize PNA agent-directed enzyme prodrug therapy, wherein a PNA agent is linked to an enzyme and injected in a subject, resulting in selective binding of the enzyme to tumor-associated or metastatic genes. Subsequently, a prodrug is administered to the subject. The prodrug is converted to its active form by the enzyme only within or nearby the cells. Selectivity is achieved by the specificity of the PNA agents and by delaying prodrug administration until there is a large differential between virus infected cells and normal tissue enzyme levels. Virus infected cells may also be targeted with the genes encoding for prodrug activating enzymes. This approach has been called virus-directed enzyme prodrug therapy (VDEPT) or more generally GDEPT (gene-directed enzyme prodrug therapy and has shown good results in laboratory systems. Other versions of directed enzyme prodrug therapy include PDEPT (polymer-directed enzyme prodrug therapy), LEAPT (lectin-directed enzyme-activated prodrug therapy), and CDEPT (clostridial-directed enzyme prodrug therapy).

Examples of prodrug activating enzymes include, but are not limited to, nitroreductase, cytochrome P450, purine-nucleoside phosphorylase, thymidine kinase, alkaline phosphatase, β-glucuronidase, carboxypeptidase, penicillin amidase, β-lactamase, cytosine deaminase, and methionine y-lyase.

In some embodiments, a therapeutic (e.g., anti-viral) agent includes a conjugate of one or more PNA agents and an anti-viral agent.

Administration

PNA agents in accordance with the invention and pharmaceutical compositions of the present invention may be administered according to any appropriate route and regimen. In some embodiments, a route or regimen is one that has been correlated with a positive therapeutic benefit.

In some embodiments, the exact amount administered may vary from subject to subject, depending on one or more factors as is well known in the medical arts. Such factors may include, for example, one or more of species, age, general condition of the subject, the particular composition to be administered, its mode of administration, its mode of activity, the severity of disease; the activity of the specific PNA agents employed; the specific pharmaceutical composition administered; the half-life of the composition after administration; the age, body weight, general health, sex, and diet of the subject; the time of administration, route of administration, and rate of excretion of the specific compound employed; the duration of the treatment; drugs used in combination or coincidental with the specific compound employed and the like. Pharmaceutical compositions may be formulated in dosage unit form for ease of administration and uniformity of dosage. It will be understood, however, that the total daily usage of the compositions of the present invention will be decided by an attending physician within the scope of sound medical judgment.

Compositions of the present invention may be administered by any route, as will be appreciated by those skilled in the art. In some embodiments, compositions of the present invention are administered by oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, vitreal, sublingual; by intratracheal instillation, bronchial instillation, and/or inhalation; as an oral spray, nasal spray, and/or aerosol, and/or through a portal vein catheter.

In some embodiments, PNA agents in accordance with the present invention and/or pharmaceutical compositions thereof may be administered intravenously, for example, by intravenous infusion. In some embodiments, PNA agents in accordance with the present invention and/or pharmaceutical compositions thereof may be administered by intramuscular injection. In some embodiments, PNA agents in accordance with the present invention and/or pharmaceutical compositions thereof may be administered by subcutaneous injection. In some embodiments, PNA agents in accordance with the present invention and/or pharmaceutical compositions thereof may be administered via portal vein catheter. However, the invention encompasses the delivery of PNA agents in accordance with the present invention and/or pharmaceutical compositions thereof by any appropriate route taking into consideration likely advances in the sciences of drug delivery.

In some embodiments, PNA agents in accordance with the present invention and/or pharmaceutical compositions thereof may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day to obtain the desired therapeutic effect. The desired dosage may be delivered more than three times per day, three times per day, two times per day, once per day, every other day, every third day, every week, every two weeks, every three weeks, every four weeks, every two months, every six months, or every twelve months. In some embodiments, the desired dosage may be delivered using multiple administrations (e.g., two, three, four, five, six, seven, eight, nine, ten, eleven, twelve, thirteen, fourteen, or more administrations).

Prophylactic Applications

In some embodiments, PNA agents in accordance with the invention may be utilized for prophylactic applications. In some embodiments, prophylactic applications involve systems and methods for preventing, inhibiting progression of, and/or delaying the onset of a viral infection, and/or any other gene-associated condition in individuals susceptible to and/or displaying symptoms of COVID-19.

Combination Therapy

It will be appreciated that PNA agents and therapeutically active conjugates thereof in accordance with the present invention and/or pharmaceutical compositions thereof can be employed in combination therapies to aid in diagnosis and/or treatment. “In combination” is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In will be appreciated that therapeutically active agents utilized in combination may be administered together in a single composition or administered separately in different compositions. In general, each agent will be administered at a dose and/or on a time schedule determined for that agent.

The particular combination of therapies (e.g., therapeutics or procedures) to employ in a combination regimen will take into account compatibility of the desired therapeutics and/or procedures and the desired therapeutic effect to be achieved. It will also be appreciated that pharmaceutical compositions of the PNA agents disclosed herein can be employed in combination therapies (e.g., combination antiviral therapies), that is, the pharmaceutical compositions can be administered concurrently with, prior to, or subsequent to, one or more other desired therapeutic procedures.

The particular combination of therapies to employ in a combination regimen will generally take into account compatibility of the desired therapeutics and/or procedures and the desired therapeutic effect to be achieved. It will also be appreciated that the therapies employed may achieve a desired effect for the same disorder (for example, an inventive antigen may be administered concurrently with another antiviral drug), or they may achieve different effects. It will be appreciated that the therapies employed may achieve a desired effect for the same purpose (for example, PNA agents useful for treating, preventing, and/or delaying the onset of a viral infection or other disorder may be administered concurrently with another agent useful for treating, preventing, and/or delaying the onset of a viral infection or disorders), or they may achieve different effects (e.g., control of any adverse effects). The invention encompasses the delivery of pharmaceutical compositions in combination with agents that may improve their bioavailability, reduce and/or modify their metabolism, inhibit their excretion, and/or modify their distribution within the body.

In some embodiments, agents utilized in combination will be utilized at levels that do not exceed the levels at which they are utilized individually. In some embodiments, the levels utilized in combination will be lower than those utilized individually.

In some embodiments, combination therapy may involve administrations of a plurality of PNA agents directed to a single gene. In some embodiments, combination therapy can comprise a plurality of PNA agents that recognize distinct gene sequences.

Kits

The invention provides a variety of kits for conveniently and/or effectively carrying out methods in accordance with the present invention. Kits typically include one or more PNA agents.

In some embodiments, kits for use in accordance with the present invention may include one or more reference samples; instructions (e.g., for processing samples, for performing tests, for interpreting results, for administering PNA agents, for storage of PNA agents, etc.); buffers; and/or other reagents necessary for performing tests. In some embodiments, kits can include panels of PNA agents. Other components of kits may include cells, cell culture media, tissue, and/or tissue culture media.

In some embodiments, kits include a number of unit dosages of a pharmaceutical composition including PNA agents. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may include one or more vessels or containers so that certain of the individual components or reagents may be separately housed. Kits may include a means for enclosing the individual containers in relatively close confinement for commercial sale, e.g., a plastic box, in which instructions, packaging materials such as styrofoam, etc., may be enclosed.

In some embodiments, kits are used in the treatment, diagnosis, and/or prophylaxis of a subject suffering from and/or susceptible to infection by SARS-CoV-2. In some embodiments, such kits include (i) at least one PNA agent; (ii) a syringe, needle, applicator, etc. for administration of the at least one PNA agent to a subject; and (iii) instructions for use.

Methods of Use

In one embodiment, the invention provides a method for treating or reducing the risk of a SARS-CoV-2 infection including administering to a subject susceptible to or having a SARS-CoV-2 infection any one of the PNA agents described herein or any one of the pharmaceutical compositions described herein.

By “susceptible” is it meant that the subject is at risk of having a SARS-CoV-2 infection. For example, it can include subject that are exposed to the virus or that are exposed to infected subject. In various aspect, the subject has COVID-19.

In many aspect, the PNA agent includes a PNA moiety including a sequence selected from SEQ ID NO:1, SEQ ID NO:2, SEQ ID NO:3, SEQ ID NO:4 and SEQ ID NO:5. In some aspects, the PNA agent includes a moiety including a nucleic acid sequence that targets an RNA sequence selected from the group consisting of SEQ ID NO:6, SEQ ID NO:7, SEQ ID NO:8, SEQ ID NO:9 and SEQ ID NO:10.

The PNA agent described herein can reduce SARS-CoV-2-associated cytopathic effects in a cell infected by SARS-CoV-2. As used herein, the term “cytopathic effect” or “CPE” is meant to include any morphological and structural change that can happen in a cell and that are caused by viral invasion. CPE can for example include cell swelling, cell lysis and cell death. In various aspects, the PNA agents described herein reduce cell death resulting from SARS-CoV-2 infection, and therefore increases the viability of a cell infected by SARS-CoV-2 in the subject.

In one aspect, the PNA agent inhibits SARS-CoV-2-associated cytopathic effects by at least 50%. For example, the PNA agent can inhibit SARS-CoV-2-associated CPE by at least 50%, 60%, 70%, 80%, 90%, 95%, 96%, 97%, 98%, 99% or more.

The efficacy of the PNA agent to inhibit SARS-CoV-2-associated CPE can be evaluated by the amount of PNA agent sufficient to inhibit 50% of the SARS-CoV-2-associated CPE (i.e., the IC50 of the PNA agent.

In some aspects, the PNA agent inhibits SARS-CoV-2-associated CPE with a IC50 that is less than about 5 μM. For example, the IC50 of the PNA agent can be less than about 5 μM, 4, μM, 3 μM, 2 μM, 1 μM, 750 nM, 500 nM, 250 nM, 100 nM, 50 nM, 10 nM or less.

The PNA agents described herein can be administered alone or in combination with another treatment, efficient for the treatment of COVID19. For example, the PNA agent can be administered in combination with an anti-viral agent. In some aspects, the anti-viral agent is Remdesivir.

In another embodiment, the invention provides a method of reducing expression of a SARS-CoV-2 target nucleic acid sequence in a cell including contacting a cell in which the target is expressed with at least one of the PNA agents described herein, determining a level or activity of the target in the cell when the PNA agent is present as compared with a target level or activity observed under otherwise comparable conditions when the PNA agent is absent; and classifying the PNA agent as a target inhibitor if the level or activity of the target is reduced when the PNA agent is present as compared with the target level or activity in the absence of the PNA agent, thereby reducing expression of the SARS-CoV-2 target nucleic acid sequence.

In one aspect, the method further includes detecting a viral load of SARS-CoV-2.

In another embodiment, the invention provides a method for identifying and/or characterizing a PNA agent as an inhibitor of a target nucleic acid sequence including contacting a SARS-CoV-2 target nucleic acid sequence with at least one PNA agent; determining a level or activity of the target sequence in a system when the PNA agent is present as compared with a target reference level or activity under otherwise comparable conditions when is the PNA agent is absent; and classifying the PNA agent as a target inhibitor if the level or activity of the target is reduced when the PNA agent is present as compared with the target level or activity when the PNA agent is absent, thereby identifying and/or characterizing the PNA agent as an inhibitor or a target nucleic acid sequence.

In one aspect, determining the level or activity of the target includes determining a target RNA level of expression. In another aspect, determining the level or activity of the target includes determining a target protein level.

Determining a target RNA level and/or a target protein level can include any of the methods know in the art to determine a RNA and/or protein level. Non-limiting examples of methods for determining a target RNA level include Northern blot analysis, nuclease protection assays (NPA), in situ hybridization, and reverse transcription-polymerase chain reaction (RT-PCR). Non-limiting examples of methods for determining a target protein level include enzyme-linked immunosorbent assay (ELISA) and related assays, western blot analysis, and mass spectrometry.

In some aspect, a reduction in the level or activity of the target includes a greater than 50-100% reduction of target activity. For example, a reduction in the level or activity of the target includes a 40%, 50%, 60%, 70%, 80%, 90%, 95%, 96%, 97%, 98%, 99% or more reduction in the target activity.

In other aspects, a reduction in the level or activity of the target includes a greater than 30% reduction of target levels. For example, a reduction in the level or activity of the target includes a 20%, 30%, 40%, 50%, 60%, 70%, 80%, 90%, 95%, 96%, 97%, 98%, 99% or more reduction in the target level.

Determining the level or activity of the target RNA or protein can indicate the efficacy of the PNA agent to inhibit said target expression and/or activity. The PNA agents described herein target SARS-CoV-2 nucleic acids. A PNA agent that reduces SARS-CoV-2 target RNA or protein levels and/or activity in an inhibitor PNA agent. By reducing SARS-CoV-2 target RNA or protein levels and/or activity, the PNA agent can reduce cell death associated with SARS-CoV-2 infection. therefore, determining the level or activity of the target RNA or protein can indicate the effect of the PNA on to cell viability. In some aspects, an inhibitor PNA agent reduces level or activity of the target RNA or protein in a cell and increases cell viability.

In some aspects, a reduction in the level or activity of the target corresponds to a greater than about 90% increase in cell viability. For example, a reduction in the level or activity of the target can correspond to an increase in cell viability greater than about 70%, 80%, 90%, 95%, 96%, 97%, 98%, 99% or more.

Depending on the number of cells in a tissue or organism that are infected and that die from a SARS-CoV-2 infection, a SARS-CoV-2 infection can lead to the death of a tissue and/or of an organism. By reducing the level or activity of a target RNA and/or protein, a PNA agent can increase cell viability. Therefore, in some aspects, the level or activity of the target corresponds to survival of the organism. In some aspects, an inhibitor PNA agent reduces level or activity of the target RNA or protein in a cell, increases cell viability, and increase survival of the tissue or organism.

In some aspects, a reduction in the level or activity of the target includes a greater than 50% increase in survival of the organism. For example, a reduction in the level or activity of the target can correspond to an increase in the organism survival greater than about 40%, 50%, 60%, 70%, 80%, 90%, 95%, 96%, 97%, 98%, 99% or more.

As used herein, the term “system” is meant to include any support in which the level of target RNA and or protein can be determined to evaluate the inhibitory effect of a PNA agent. A system can include an in vitro system, such as a cell in culture, or an in vivo system such as a cell, a tissue, or an organism (including non-human mammal and human). In various aspects, when the system is tissue, or an organism, it is meant a tissue or an organism comprising cells. In various aspects, the cells include SARS-CoV-2 virus.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### In Vitro Antiviral Testing

A key step in drug discovery is screening to evaluate antiviral activity. After determining the appropriate compounds and mechanisms, the next step is to perform cytotoxicity analysis of the compounds to ensure your efficacy data are meaningful and within a reasonable therapeutic window. Here is a short list of antiviral assays and virus replication assays useful in the present invention for evaluating SARS-CoV-2 PNAs.

One of skill in the art will be able to identify suitable virus replication and infection assays. For example, the following publication provides such assays and is herein incorporated by reference in its entirety:

https://www.essenbioscience.com/en/communications/immunology/viral-replication-and-infection-assays/

Further, Rumlova and Ruml, Biotechnology Advances 36 (218) 557-576, which is herein incorporated by reference in its entirety, is provided for disclosure of method for testing for antiviral drugs, and in particular drugs effective for SARS-CoV-2 infection.

Links to protein sequences, herein incorporated by reference in their entirety. One of skill in the art can determine suitable sequences for use in the PNAs of the present invention based on the protein sequences provided. It is known in the art that the virus has a single stranded positive RNA genome, thereby allowing for target identification:

Spike (S): uniprot.org/uniprot/P0DTC2
Nucleoprotein (N): uniprot.org/uniprot/P0DTC9
Envelop (E): uniprot.org/uniprot/P0DTC4
Membrane (M): uniprot.org/uniprot/P0DTC5

Various virus replication assays are known in the art, including but not limited to, by way of example:

Cytopathic effect (CPE) inhibition assay. CPE is morphological changes in cells caused by cytopathogenic virus infection. CPE assay is used to evaluate test articles' ability to inhibit CPE. This is the most cost-effective and time-efficient assay we offer for high throughput screening of overall antiviral activity. For non-cytopathic viruses we offer cell-based enzyme-linked immunosorbent assay (ELISA) or quantitative real-time Polymerase Chain Reaction (PCR) assay.

Cell-based ELISA. Cell-based ELISA measures reduction of viral antigen in infected cells using anti-virus monoclonal antibody. The abundance of viral protein in infected cells treated with the test article compared to that of the untreated control is used as a measure of antiviral activity.

qPCR assay. qPCR assay uses oligonucleotide primers and a probe amplifying virus-specific target sequence to detect the presence of virus nucleic acids. Reduction of virus nucleic acid in infected cells is used an indicator of a test article's antiviral efficacy.

Plaque reduction assay. Infectious virus particles multiply in cells and result in circular zones of infected regions, plaques. Plaque reduction assay measures the plaque forming efficiency of a virus in the presence of different concentrations of a test article. Plaque reduction neutralization test (PRNT), a variation of this assay, is considered the gold standard for detecting neutralizing antibodies to certain viruses (i.e., flavivirus).

Yield reduction assay. Yield reduction assay is a labor-intensive but powerful technique for evaluating a compound's antiviral efficacy. The three-step assay involves: infecting cells in the presence of different concentrations of the test article; collecting the cells or cell culture supernatants after a cycle of virus replication; and determining virus titers by plaque assay, TCID50, or quantitative real-time PCR.

Antibody-dependent enhancement (ADE) assay. ADE occurs when non-neutralizing or sub-neutralizing antiviral proteins facilitate virus entry into host cells leading to enhanced infectivity. ADE, which has been observed in viruses such as Dengue and Influenza, poses a challenge in vaccine development. Using flow cytometry, plaque assay or qPCR, this assay evaluates the ADE effect of test articles on virus infection in Fc receptor bearing cells.

Quantitative suspension test. This test is used to evaluate virucidal activity of chemical disinfectants within a given contact time in suspension. Generally, a 4 log 10 reduction in virus titer (99.9% inactivation) is an indicator of a disinfectant's virucidal properties detected under the test conditions.

### Example 2

### Material and Methods

Screening Strategy

A cell-based assay was employed to measure the cytopathic effect (CPE) of the virus infecting Vero E6 host cells. The CPE reduction assay is widely used assay format to screen for antiviral agents because of its ease of use in high throughput screening (HTS). In this assay, host cells infected with virus die as a consequence of the virus hijacking the cellular mechanisms for genome replication. The CPE reduction assay indirectly monitors the effect of antiviral agents acting through various molecular mechanisms by measuring the viability of host cells three days after inoculation with virus. Anti-viral compounds are identified as those that protect the host cells from the cytopathic effect of the virus, thereby increasing viability.

Compound Preparation

Compound stock solutions (80 μL of 150 μM solution in water) were transferred into wells of an empty ECHO plate (stock plate). Compounds were diluted 2-fold by transferring 40 μL of each stock sample into an adjacent well containing 40 μL water and mixing. This process was repeated to create 8 more wells of serially diluted sample, each well containing a 2-fold diluted sample of the previous well. A 600 nL aliquot of each diluted sample was dispensed into corresponding wells of assay ready plates using an ECHO555 acoustic liquid handling system. The final assay concentration range was 3.0-0.006 μM.

Method for Measuring Antiviral Effect of Compounds

Vero E6 cells selected for expression of the SARS CoV receptor (ACE2; angiotensin-converting enzyme 2) were used for the CPE assay. Cells were grown in MEM supplemented with 10% HI FBS and harvested and suspended in MEM, 1% Pen/Strep, supplemented with 2% HI FBS on the day of assay. Assay ready plates pre-drugged with test compounds were prepared in the BSL-2 lab by adding 5 μL assay media to each well. The plates and cells were then passed into the BSL-3 facility. Cells were batch inoculated with SARS CoV-2 (USA_WA1/2020; M.O.I.˜0.002) which results in 5% cell viability 72 hours post infection. A 25 μL aliquot of virus inoculated cells (4,000 Vero E6 cells/well) was added to each well in columns 3-24 of the assay plates. The wells in columns 23-24 contained only virus infected cells for the 0% CPE reduction controls. Prior to virus inoculation, a 25 μL aliquot of cells was added to columns 1-2 of each plate for the cell only 100% CPE reduction controls. After incubating plates at 37° C./5% CO₂ and 90% humidity for 72 hours, 30 μL of Cell Titer-Glo (Promega) was added to each well. Luminescence was read using a BMG CLARIOstar plate reader following incubation at room temperature for 10 minutes to measure cell viability. Plates were sealed with a clear cover and surface decontaminated prior to luminescence reading.

Method for Measuring Cytotoxic Effect of Compounds

Compound cytotoxicity was assessed in a BSL-2 counter screen. Host cells in media were added in 25 μl aliquots (4,000 cells/well) to each well of assay ready plates prepared with test compounds as above. Cells only (100% viability) and cells treated with hyamine at 100 μM final concentration (0% viability) serve as the high and low signal controls, respectively, for cytotoxic effect in the assay. DMSO was maintained at a constant concentration for all wells as dictated by the dilution factor of stock test compound concentrations. After incubating plates at 37° C./5% CO2 and 90% humidity for 72 hours, 30 μl Cell Titer-Glo (Promega) was added to each well. Luminescence was read using a BMG PHERAstar plate reader following incubation at room temperature for 10 minutes to measure cell viability.

Data Analysis

For all assays the raw data from plate readers were imported into ActivityBase where values were associated with compound IDs and test concentrations.

For the antiviral CPE reduction assay, raw signal values are converted to % CPE reduction by the following formula:

% CPE reduction=100×(test cmpd value−mean value infected cell controls)/(mean value uninfected cell controls−mean value infected cell controls).

For the cell viability assay measuring compound cytotoxicity, % cell viability is calculated as follows:

% viability=100*(test cmpd value−mean low signal control)/(mean high signal control−mean low signal control).

EC₅₀ and CC₅₀ values were calculated from a four-parameter logistic fit of data using the Xlfit module of ActivityBase.

### Example 3

### Evaluation of the Efficacy of Anti-SARS-Cov-2 PNAs Agents

The efficacy of 5 different anti-SARS-CoV-2 PNAs (AB01971749, AB01971744, AB01971754, AB01971748, and AB01971753), having the PNA sequences and target sequences as shown in Table 1, was evaluated in Vero E6 cells by measuring the % CPE reduction, and cell viability as described in Example 2.

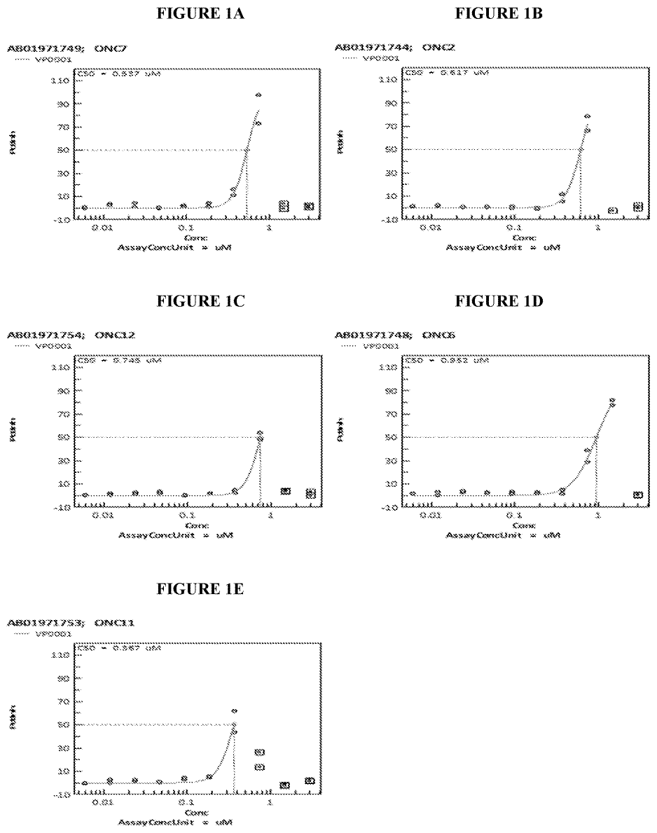

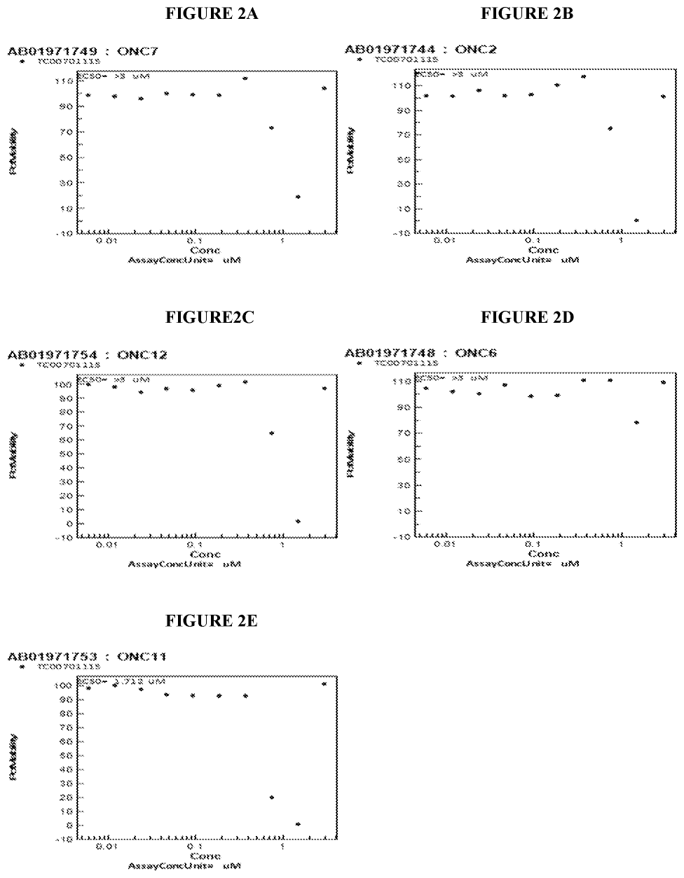

As illustrated in FIGS. 1A-1E, and as further detailed in Table 2, the PNAs showed a percent CPE inhibition ranging from 53 to 97%, while displaying an IC50 ranging from 0.37 to 0.95 μM. The maximal percent inhibition was observed at PNA's concentrations within close range to the IC50, which translated in limited toxicity associated with the PNAs (see FIGS. 2A-2E). Further, the target sequences included both positive (SEQ ID NO: 8 and 10) and negative (SEQ ID NO: 6, 7, 9) strand targets to illustrate that the target is not limited to inhibition of the viral genome or mRNA for translation specifically.

TABLE 1
Anti-SARS-CoV-2 PNAs sequences and target sequences

| Compound ID | PNA sequences | SEQ ID NO: | target sequences | SEQ ID NO: |
|---|---|---|---|---|
| AB01971749 | AAGTTGGTTGGTTTGTT | SEQ ID NO: 1 | AACAAACCAACCAACTT | SEQ ID NO: 6 |
| AB01971744 | GTTGGTTTGTTACCTG | SEQ ID NO: 2 | CAGGTAACAAACCAAC | SEQ ID NO: 7 |
| AB01971754 | ACCAACCAACTTTCGA | SEQ ID NO: 3 | TCGAAAGTTGGTTGGT | SEQ ID NO: 8 |
| AB01971748 | TTGGTTGGTTTGTT | SEQ ID NO: 4 | AACAAACCAACCAA | SEQ ID NO: 9 |
| AB01971753 | CCAACTTTCGATCTCTT | SEQ ID NO: 5 | AAGAGATCGAAAGTTGG | SEQ ID NO: 10 |

TABLE 2
Anti-SARS-CoV-2 PNAs efficacy summary

| Compound | IC50 | Max % | Concentration at |
|---|---|---|---|
| ID | (uM) | Inhibition | Max % Inhibition |
| AB01971749 | 0.54 | 97.37 | 0.75 |
| AB01971744 | 0.62 | 78.26 | 0.75 |
| AB01971754 | 0.75 | 53.70 | 0.75 |
| AB01971748 | 0.95 | 81.65 | 1.50 |
| AB01971753 | 0.37 | 61.51 | 0.38 |

Although the invention has been described with reference to the above example, it will be understood that modifications and variations are encompassed within the spirit and scope of the invention. Accordingly, the invention is limited only by the following claims.

SEQUENCES

| | |
|---|---|
| AB01971749, PNA sequence | SEQ ID NO: 1 |
| AAGTTGGTTGGTTTGTT | |
| AB01971744, PNA sequence | SEQ ID NO: 2 |
| GTTGGTTTGTTACCTG | |
| AB01971754, PNA sequence | SEQ ID NO: 3 |
| ACCAACCAACTTTCGA | |
| AB01971748, PNA sequence | SEQ ID NO: 4 |
| TTGGTTGGTTTGTT | |
| AB01971753, PNA sequence | SEQ ID NO: 5 |
| CCAACTTTCGATCTCTT | |
| AB01971749, target sequence | SEQ ID NO: 6 |
| AACAAACCAACCAACTT | |
| AB01971744, target sequence | SEQ ID NO: 7 |
| CAGGTAACAAACCAAC | |
| AB01971754, target sequence | SEQ ID NO: 8 |
| TCGAAAGTTGGTTGGT | |
| AB01971748, target sequence | SEQ ID NO: 9 |
| AACAAACCAACCAA | |
| AB01971753, target sequence | SEQ ID NO: 10 |
| AAGAGATCGAAAGTTGG | |

1. A peptide nucleic acid (PNA) agent comprising:
a PNA moiety comprising a nucleic acid sequence that targets a SARS-CoV-2 nucleic acid sequence;
a first cationic and hydrophobic peptide at the N-terminus of the PNA moiety, wherein the first peptide comprises lysine residues; and
a second cationic and hydrophobic peptide at the C-terminus of the PNA moiety, wherein the second peptide comprises lysine residues.

2. The PNA agent of claim 1, wherein at least one of the lysine residues comprises a palmitoyl side chain moiety.

3. (canceled)

4. The PNA agent of claim 1, wherein the PNA agent comprises a sequence that contains less than 60% purines.

5. The PNA agent of claim 1, wherein the PNA moiety comprises a sequence that targets an RNA sequence of SARS-CoV-2 virus.

6. (canceled)

7. The PNA agent of claim 1, wherein the PNA moiety comprises a sequence selected from SEQ ID NO:1, SEQ ID NO:2, SEQ ID NO:3, SEQ ID NO:4 and SEQ ID NO:5.

8. The PNA agent of claim 1, wherein the PNA moiety comprises a nucleic acid sequence that targets an RNA sequence selected from the group consisting of SEQ ID NO:6, SEQ ID NO:7, SEQ ID NO:8, SEQ ID NO:9 and SEQ ID NO:10.

9-10. (canceled)

11. The PNA agent of claim 5, wherein the nucleic acid sequence is a SARS-CoV-2 nucleic acid sequence encoding a structural protein or an open reading frame (orf) protein.

12. The PNA agent of claim 11, wherein the SARS-CoV-2 nucleic acid sequence encodes a structural protein selected from a Spike (S) protein, Nucleocapsid (N) protein, Envelope (E) protein, or Membrane (M) protein.

13. (canceled)

14. A pharmaceutical composition comprising the PNA agent of claim 1 and pharmaceutically acceptable carrier.

15. A method for treating or reducing the risk of a SARS-CoV-2 infection comprising:
administering to a subject susceptible to or having a SARS-CoV-2 infection the PNA agent of claim 1.

16. (canceled)

17. The method of claim 15, wherein the PNA agent comprises a PNA moiety comprising a sequence selected from SEQ ID NO:1, SEQ ID NO:2, SEQ ID NO:3, SEQ ID NO:4 and SEQ ID NO:5.

18. (canceled)

19. The method of claim 15, wherein the PNA agent increases the viability of a cell infected by SARS-CoV-2 in the subject.

20. The method of claim 15, wherein the PNA agent reduces SARS-CoV-2-associated cytopathic effects in a cell infected by SARS-CoV-2 in the subject.

21. (canceled)

22. The method of claim 15, wherein the PNA agent inhibits SARS-CoV-2-associated cytopathic effects by at least 50%.

23-24. (canceled)

25. A method of reducing expression of a SARS-CoV-2 target nucleic acid sequence in a cell comprising:
contacting a cell in which the target is expressed with at least one PNA agent of claim 1;
determining a level or activity of the target in the cell when the PNA agent is present as compared with a target level or activity observed under otherwise comparable conditions when the PNA agent is absent; and
classifying the PNA agent as a target inhibitor if the level or activity of the target is reduced when the PNA agent is present as compared with the target level or activity in the absence of the PNA agent, thereby reducing expression of the SARS-CoV-2 target nucleic acid sequence.

26. The method of claim 25, further comprising detecting a viral load of SARS-CoV-2.

27-28. (canceled)

29. The method of claim 25, wherein the PNA agent comprises a PNA moiety comprising a sequence selected from SEQ ID NO:1, SEQ ID NO:2, SEQ ID NO:3, SEQ ID NO:4 and SEQ ID NO:5.

30-34. (canceled)

35. A method for identifying and/or characterizing a peptide nucleic acid (PNA) agent as an inhibitor of a target nucleic acid sequence comprising:
contacting a SARS-CoV-2 target nucleic acid sequence with at least one PNA agent;
determining a level or activity of the target sequence in a system when the PNA agent is present as compared with a target reference level or activity under otherwise comparable conditions when is the PNA agent is absent; and
classifying the PNA agent as a target inhibitor if the level or activity of the target is reduced when the PNA agent is present as compared with the target level or activity when the PNA agent is absent, thereby identifying and/or characterizing the PNA agent as an inhibitor or a target nucleic acid sequence.

36. The method of claim 35, wherein determining the level or activity of the target comprises determining a target RNA level of expression or a target protein level.

37-52. (canceled)

53. An isolated nucleic acid sequence selected from SEQ ID NO:1-10.

54-55. (canceled)
